(19) United States
(12) Patent Application Publication
(10) Pub. No.: US 2015/0247066 A1
(43) Pub. Date: Sep. 3, 2015

(54) Sports patches and sports tapes configured to be adhered to the skin of a user

(71) Applicant: John C. Ramirez, Redlands, CA (US)

(72) Inventor: John C. Ramirez, Redlands, CA (US)

(21) Appl. No.: 14/544,855

(22) Filed: Feb. 25, 2015

Related U.S. Application Data
(63) Continuation of application No. 13/573,479, filed on Sep. 18, 2012, now abandoned.

(51) Int. Cl. C09J 7/04; A61F 13/00; A61F 13/02

(52) U.S. Cl. CPC C09J 7/04; C09J 7/046; A61F 13/02; A61F 13/00063; C09J 2201/122; C09J 2201/16; C09J 2201/20; C09J 2400/263

(57) ABSTRACT

According to the various features, characteristics and embodiments of the present invention which will become apparent as the description thereof proceeds, the present invention provides attachable grip enhancers for playing the game of various sports, by improving sports tape that are adapted to adhere to the skin of an individual, and uses and methods thereof. The present invention and its embodiments can, among other things, enhance the overall performance in arm tasks conducted by the wrist, forearm and/or bicep areas, as well as hand tasks by, among other things, creating a high coefficient of friction on said areas. Embodiments can additionally benefit users to, among other things, protect an injury, and/or protect from injury, by offering embodiments with a therapeutic additive and/or shock-absorbing member.

### FIELD OF THE INVENTION

The present invention relates to general use apparatus, and uses thereof, used in playing the game of various sports. The present invention and its embodiments can, among other things, enhance the overall performance in arm tasks conducted by the wrist, forearm and/or bicep areas, as well as hand tasks, by offering a gripping means on the top surface of said embodiments. These embodiments are adhered to an individual by an adhesive means on the bottom surface, thereby providing, among other things, said individual with enhanced grip and/or control capabilities. General use embodiments may also be secondarily adhered to something already on the skin, such as a band aid, bandage, gauze, sport pre-wraps, or an arm pad, and the like. Embodiments may additionally benefit users to, among other things, protect an injury, and/or protect from injury, by also offering a therapeutic additive and/or a shock-absorbing member.

### BACKGROUND OF THE INVENTION

Grip and control enhancers have come a long way in assisting one to complete a task or goal. Many tasks, for example, may be divided into arm tasks and hand tasks, and successful completion of said tasks often require the ability to properly grip or control an object.

Arm task grip enhancers could assist an individual complete a sport objective. An important goal in playing sports is to win. Often that means proper play execution, proper form in the sports fundamentals and especially good ball control. Many of these tasks are performed by the hand, but are often also performed, to some degree, with parts of the arm (the wrists, forearm, elbow and bicep areas). Ball control and therefore proper play execution depend on adequately using parts of the forearm, elbow and/or bicep areas to handle, strike, control or otherwise maintain possession. Inadequate play execution can result in inconsistencies and turnovers, both long standing problems in many sports, and can often determine the outcome of a game.

In the sport of football, for example, lack of play execution is often categorized by turnovers, fumbles and incomplete passes. This is of particular concern to those players that have to control a football such as running backs, receivers, tight ends, kick returners, punt returners and even quarterbacks. Athletes that play any of these positions are often asked, in some way, to run, catch, throw or otherwise control a football. Successful play execution—which in the case of football includes minimizing fumble, incompletes and inconsistencies—can often be the difference in the successful outcome of a team's objective. Creating and maintaining a solid and stable control of the ball is therefore essential in proper play execution and performance.

In football, as in many other sports, controlling a ball is often done not just by using their hands, but by using other parts of the body as well.

A football running back, for instance, might be particularly concerned with not fumbling the ball. A running back's performance is measured not only by his yards per carry but also in his ability to minimize his fumbles. Unfortunately, one need only view the statistics to see that fumbles persist as an insoluble problem, even at the professional level today.

Part of the problem lie in the seemingly inherently unstable way a player controls and cradles a ball when running. Proper ball handling technique is to grab one end of the football with your hand, and then resting the ball on the forearm of the same arm. As you begin to run with the ball, you may also place the opposite end of the ball (the end that is not being held by the hand) in the inside elbow area, between the forearm and bicep, and the ball is almost always touching the wrist area. Although prior art exists to increase grip around the hand in the form of gloves, these gloves do little to increase ones grip in the general arm area, such as the wrist, forearm, elbow and bicep areas. The development of a product which could enhance one's grip-enhancing abilities on, say the forearm area, would therefore go a long way to solving this significant problem.

Football players who catch a football (hereinafter called ‘receivers’) might be particularly concerned with making a catch and completing a reception, and being able to control a ball with the arm area. Enough skill and precision must take place in order to get the ball from the quarterback to a receiver; timing, stable footing, and protecting the ball just to name a few.

When a football receiver first catches a ball, he usually brings the ball onto his arm, thus holding the ball with the hand, as well as the forearm and elbow area. Prior art is lacking that enhances the elbow grip or the elbow area, an area that plays a significant role in proper play execution. Passing the ball is a significant part of the sport of football, sometimes throwing as much as 103 times in a single game (Seattle vs. San Diego, 2002). Thus, developing a solution to enhance one's ability of better controlling a catch and completing a pass reception would substantially impact the sport.

Additionally, inconsistencies, or incompletes, often arise when a ‘stress factor’ is introduced once a receiver first touches and places the ball on their arm area. This problem is so pronounced that many instant replay situations revolve around seeing whether the ball moves even slightly in the receivers arm—including the forearm which has no real grip enhancers. Again, because prior art (such as gloves) primarily provide grip enhancing support only around the hands, and not around other areas of the arm responsible for holding and controlling the football from moving, the receiver can often have great difficulty in keeping the football from moving. As such, an unrecognized problem currently exists because it is very difficult to grip a ball with parts of the arm.

Clearly, maintaining good ball control is important. In football, unstable or weak ball control can, among other things increase fumbles, increase incompletes and thereby increase turnovers and decrease performance.

There have been some attempts through the years to solve the problems of inconsistencies and turnovers in the sport of football. For example, changes have been made to the actual football in order to make the ball easier to handle. Changes to the shape and size, as well as the addition of grip enhancing materials to the ball—such as the addition of PVC (PolyVinyl Chloride) dots—have made it possible to make the ball more grippable. The ability of the player to maintain control of the football was still problematic because of the lack of any grip enhancing device for the player to use; devices that could be placed on the arm such that the player could now more significantly control a ball with his arm, thereby creating an overall grip of the football throughout the football. As a result of this unmet need, inconsistencies and turnovers were still high in the sport.

The introduction and subsequent proliferation of the use of gloves found some success but even with these advancements, however, fumbles and incompletes still persist today. For one, whereas a running back who uses a grip enhancing glove will be better able to properly grip a football by using his hands, his hand, and therefore the grip enhancing device, only cover the front part of the ball, leaving the rest of the ball, and therefore the overall grip, still significantly unstable. One need only add a stress factor and this currently unstable hold on the ball can easily result in a fumble.

Good ball control is so important in football that inventions were created and widely used to enhance the gripping abilities of an individual's hand. Unfortunately, significant grip improvements stopped with the hand gloves. Improvements have lacked in assisting the rest of the arm to better handle a ball. Improvements have lacked in providing enhanced gripping support around the forearm area, an area that plays a major role in maintaining control of a ball when a receiver is completing a reception. Improvements have lacked in offering a player the ability to achieve an enhanced grip capability throughout the general arm area, where the ball touches the player (such as the forearm, elbow and bicep area). Improvements have lacked in increasing one's grip around the wrist area (an area that almost always touches the ball when cradling the ball), nor have there been advances to significantly increase ball control around the elbow or bicep areas.

Providing such a device would certainly allow a player to have a much stronger grip throughout and around the ball, to create a more stable overall handle on the ball, and therefore to significantly advance arm task performance and play execution. Not only would new art offer benefits to running backs, receivers, tight ends and quarterbacks, but they could also increase the performances of kick returners and punt returners, who have to run with the football.

These grip-enhancing shortcomings make it no surprise that there were a total of 731 fumbles, and the highest quarterback completion rating was less than 70 percent, in the 2010 NFL Season (Official Stat Book of the NFL, 2011). Given the fact that fumbles and incomplete passes persist at the professional level and therefore certainly at the collegiate and amateur levels, one can see that past attempts to solve these problems have had limited success.

An example of another sport in need of grip enhancers for the arm area is VOLLEYBALL. Here too, ball control is crucial to play performance. Play execution often depends on one's ability to control a ball with multiple parts of the arm, but especially the forearm. Although there are forearm, elbow and even bicep devices that could be used for protection, or simply for aesthetic purposes, no prior art exists that can adequately enhance ball control or grip in said areas.

When an opponent strikes a volleyball to your team's side of the net, the first player to normally touch a ball—when the ball isn't blocked at the net—generally controls the ball with her or his forearm(s). With that first touch contact, one must be able to stabilize the ball, and then usually pass the ball to a teammate. Unfortunately, this current method of passing a volleyball can result in inconsistent results. First, the initial contact control is generally with the skin of the forearm of the player, and without any grip-enhancing device on the forearm, it can become very difficult to redirect a volleyball with consistent precision. Although the initial contact happens very fast, a control-enhancing mechanism which could be firmly attached to the arm area would have a significant impact on the sport. Additionally, a more general reason for inconsistencies is in one's inability to grip with one's forearm. Without providing the forearm with some way to better grip a volleyball, inconsistencies in the sport of volleyball will continue to be a long-standing problem.

More generally, there are clear indications that an entirely new market exists for targeted grip enhancers that could be adhered (using an adhesive means) only on select areas of the arm area.

Not only are there significant needs for grip-enhancing devices for the arm area, in particular the wrist, forearm, elbow and bicep areas, but there is also a significant need for better grip-enhancing devices for the hand area as well. Although offering some advantages, using prior art—such as gloves for the hand—creates disadvantages that often force an individual to choose not to use any grip enhancing device at all.

There are several non-sport activities, for example, in need of targeted hand grip enhancers for general use.

As an example, there are several indoor activities where there is an unrecognized need for hand task enhancers. Activities requiring prolonged hand movements, such as sweeping, mopping, and general house cleaning often require one to grip and/or otherwise control an object. Many choose to conduct these tasks bare-handed often because they don't need all the protection from injury or from grime usually associated with having to use a glove. Many brooms, mops and other household devices however, do not have a grip enhancing component on their handles, leaving an opportunity to provide a non-industrial strength grip enhancer, such as perhaps a gripping means for the hand, which can enhance only the part of the hand that requires grip enhancers without encumbering the rest of the hand. This could result in significant increase in overall ease of use and therefore increased cleaning performance.

Many basic home repair activities requiring tools, such as hammers and wrenches, are often completed using bare hands (especially when the repair work is done indoors). Many individuals choose not to spend the money on expensive construction gloves, and instead choose to forgo any extra grip enhancers—or protection—and complete the task bare-handed. A result, with prolonged use, often can be the development of blisters on parts of the hand areas. There therefore is an opportunity to provide a relatively more cost effective and convenient solution whereby an individual could complete these hand tasks. Offering a product, such as a new type of tape which could be adhered to an individual's skin or onto a skin-contact product already on the human body (such as bandages or pre-wraps), could therefore also protect an individual from injury (such as from getting a blister).

The use of gloves has other drawbacks, especially when water is involved. Unless one buys expensive swimmers-type gloves that are waterproof, one is usually forced to go without any grip enhancers when conducting activities involving water. Providing, therefore, a cost-effective waterproof grip enhancer would certainly meet a currently unmet need.

Improved art is also desired to support outdoor yard work. Quite often one chooses to use gloves for outdoor yard work. Although it does protect ones hand from injury and grime, at times one finds that they have to take off the gloves momentarily, especially during the hot summer months because of the perspiration building up on the hand from the use of gloves. This inconvenient and unnecessary problem is created because of lack of ventilation in many yard gloves. When the yard work requires intense activity, then the gloves are worth the hassle, but very often the yard work is not very demanding. For example, when one has to control a shovel or rake, the work is often not very demanding but it is very repetitive. The choice that one faces is to either wear a glove for the added grip, but then have to stop a few times to take the gloves off and dry your hands, or choose not to use a glove at all. One result, at times, is that one chooses to wear a glove because of a lack of an alternative that might possess many of the advantages of wearing a glove but without any of the disadvantages.

More generally, there are clear indications that an entirely new market exists for targeted grip enhancers that could be adhered (using an adhesive) on select areas on the hand, leaving other parts of the hand uncovered. These grip enhancers could be adhered to the skin of an individual or could be attached to something already on an individual (such as over a bandage). These new grip enhancers would also find success if they also offered a therapeutic additive layer to protect an injury or to help protect from injury.

Another particular area where prior art poses significant disadvantages is in any sport where a player's success depends on her ability to both grip as well as feel a ball or object. These players often have to choose between wearing a glove thereby enhancing grip but losing significant feel, or going without a glove thereby maintaining maximum feel but missing the opportunity to enhance one's grip.

In football for example, gloves can be used to enhance performance. The use of gloves in football is so widespread that nearly every football player uses them, with the notable exception of football quarterbacks. You rarely see a quarterback wear gloves, even if just to keep warm. Most quarterbacks choose to play football without gloves. This is largely because prior art consists of generic full-fingered gloves which are uncomfortable and burdensome on a quarterback's dominant (throwing) hand, particularly on those fingers a quarterback places over the football laces. In addition, the full-fingered gloves prevent a quarterback to have any ‘feel’ of the ball, a necessary element in a quarterback's success.

Playing the position of quarterback without any grip enhancing mechanism, however, can also be an inferior choice. The website Wikihow.com, provides a good description of the conventional way to hold and throw a football: “Throwing the football is simple. Put your non-throwing side foot in front of you. Have your Pinkie, Ring and Middle fingers around the laces with your Index [Forefinger] finger on the strap. Put the other hand up on the ball. Put the ball up by your ear. Twist your hips toward the front foot. Throw the ball at the receiver.” Whereas, the fingers over the laces have a solid grip on the ball—primarily due to the football laces on the ball—the two fingers off the laces (forefinger and thumb) are virtually unsupported and therefore have a relatively weaker grip, creating a weak overall grip on the football.

This weak overall grip becomes more pronounced when added stress is placed on the Thumb or Forefinger. When a quarterback, intending to pass the football, for example, suddenly has to scramble, or if the quarterback ‘pumps’ the ball (goes through all the motions and speed of throwing the ball but doesn't actually release the ball), the grip strength of the Thumb and Forefinger can determine whether or not a quarterback fumbles the ball. Also, if one performs a simple test and wets his/her dominant hand, and then grabs and pumps a football, the Forefinger and Thumb will often move or slip. On a wet football field, during extreme weather conditions (hot or cold), that weaker or looser grip makes for a much more difficult completed pass, less success at throwing a spiral, and inconsistency and inaccuracy in passing.

Under the ‘tips’ section of Wikihow.com, it further describes proper football throwing form: “A proper throw will feel like it's only utilizing the Thumb, Index [Forefinger], and Middle finger. Good release will ‘roll’ off of your Index and Middle finger, to impart more spin; you may snap your wrist through as you follow through to the hip. The other three fingers on your hand stabilize the ball as its being flung. They should not be used to impart spin on the ball. The most important finger to throwing a spiral is the Index finger; it is the finger that holds the most leverage in putting spin on the ball.”

This need to ‘feel’ a ball with a hand has therefore resulted in quarterbacks having to make a difficult choice. Although clearly these players would benefit from added grip enhancers on the throwing (dominant) hand—especially supporting the forefinger—prior art (in the form of gloves) force a quarterback to choose between all feel or no feel. Virtually all quarterbacks have chosen to maintain feel and sacrifice the ability to better grip the football. It is no surprise that quarterback fumbles remain a significant problem in football, even at the highest performance levels, and currently remains an insoluble problem in the sport for amateurs and professionals alike.

Individuals who play basketball also have to ‘feel’ and grip a ball to perform properly, and although they too could significantly enhance performance in controlling a ball, prior art forces them to choose all feel as well, and go without any type of grip-enhancers at all. This insoluble problem therefore also exists in playing the sport of basketball, and these players would substantially benefit from developing a way to maintain feel while increasing grip capabilities in select areas of the hand. More specifically, new art is needed that could offer control enhancers in certain locations of the hand while leaving other areas of the hand uncovered and therefore being able to maintain necessary feel.

In the field of GOLF, to be sure, there exists much prior art in the form of gloves for a golfer's weak (non-dominant) hand. In fact most active golf players wear a glove on their weak hand, and go without a glove for their strong hand (if one were to go to any major store to buy golf gloves, they would be sold and packaged in singles—one glove—not sold in pairs). Gloves are prevalent in golf largely because of the role that hand grip and control play in a golfer's overall performance.

Although there exist many types of full-fingered gloves for a golfer's weak-hand, they all attempt to maximize a golfer's weak-hand grip without regard to a golfer's weak-hand feel, and hand coordination needs. It is no surprise, therefore, that prior art consists of full-fingered (all fingers are covered), closed palm (entire palm is essentially all covered) gloves. As a result, a typical golfer must rely on his/her weak-hand to provide most of the grip support, and on his strong-hand to provide all of the ‘feel’ in his golf swing. The current solution to this insoluble problem has been for virtually all golfers to use one and only one glove. This glove is always placed on the weak hand, leaving the strong hand without a glove. There is, therefore, an opportunity to invent a device or method that could offer some ‘feel’ ability for the weak-hand, without significantly diminishing that enhanced grip ability that gloves offer. This would increase overall hand control of a golfer's club swing, and therefore result in greater success in competition.

Whereas weak-hand support products seem to be crowded in the sport of Golf, there is a long existing need for a device that could offer added support for a golfer's strong-hand without significantly diminishing its ability to adequately feel the golf club. Inventing a solution to this problem could, among other things, allow for greater golf swing control and consistency, and create an entirely new market because golfers currently do not use grip-enhancers on their dominant hand, thereby changing the way that golf is played.

In Golf magazine's April 2005 article titled “Fix your grip. The wrong grip can cripple your swing—Here's the cure”, golf instructor Charlie King provides an overview of how to grip a golf club. “Good golf starts with your grip. The proper hold on the club helps you do three crucial things: Hinge your wrists, control the clubface at impact and support the club throughout the swing. Here are three simple grip tips.” As King continues, his third tip is “both hands; solid at the top. An effective grip sets the face square at the top, with the shaft parallel to the target line. You should feel most of the club's weight in your left Thumb and right Forefinger. Now you're ready to turn it loose.” Although prior art seems to be crowded in offering gloves for the weak-hand, to support and better control the club weight placed on the Thumb of the weak-hand, there remains an unmet need for added support on or around the Forefinger of the strong-hand.

Additionally, constant swinging of a golf club at real swing speeds often results in soreness on and between the Thumb and Forefinger of a golfer's strong hand (wearing no glove). This soreness can often also come from the rubbing or slipping, between the club handle and the strong-hand, suggesting a need to find a way to increase the grip—as well as protection—of a golfer's strong hand. This is especially important in the sport of golf because even the smallest of slipping—during the golf swing or upon impact of the golf ball—can create enormous inconsistencies and inaccuracies, critical issues in determining overall performance.

Consequently, there are clear indications that an entirely new market exists for targeted grip-enhancers that could be placed only on select areas of the strong (dominant) hand, leaving other parts of the strong hand free to feel. In particular there remains an unrecognized problem and an unmet need that would provide multiple benefits, such as better overall grip, more coordination with both hands, as well as some protection from, for example, any constant grip slipping, during the practice or play of golf; and in various other sports activities.

In the sport of BASKETBALL, there exists no prior art when it comes to attachable grip-enhancers to enhance the performance of over 100 million individuals who play the sport. Although there are several multisport gloves in the market today, virtually no one uses gloves when playing basketball. A primary reason why basketball players choose not to use gloves, as mentioned briefly above, is similar to the issue with football quarterbacks, basketball players often need to be able to both grip and feel the ball. Although many hand tasks require a good grip, no art currently exists that would provide these players with enhanced grip capabilities or enhanced protection by being able to cover only select parts of the hand, without having to sacrifice the critical feel ability along other parts of the hand.

One clear hand task in basketball is in shooting the basketball with the intention of making a score or basket. Conventional jump-shot shooting form requires, among other things, that the player hold the basketball largely with the fingertips of both hands, and creating a small opening—or a shooter's gap—between the ball and the palm area of the player's strong-hand. No grip enhancers exist that would increase the gripping abilities of a players fingertips and leaving the rest of the hand free to feel if the basketball is touching or violating, for example, the shooters gap.

Prior art is lacking that would provide an athlete with the ability to have enhanced control when dribbling a basketball. Proper dribbling form is to rarely, if ever, look at the ball while dribbling said ball—thus one of the critical reasons of being able to maintain high ‘feel’ ability. Without any extra grip-enhancers however, it is difficult to maintain stable control of the basketball.

A typical game—even a professional game—often can have as many as 30 turnovers (combined), so offering art that could increase ball control while dribbling, passing or even catching a basketball could significantly enhance performance by, among other things, minimizing turnovers. Minimizing turnovers, for example, could be achieved by offering better ball control while dribbling a basketball. Those players playing the position of Guard may benefit from added grip support especially because they may need to dribble, at least briefly, with either hand.

Although athletes playing the position of Forward or Center would also benefit by enhanced dribbling abilities, most of the turnovers caused by Forwards and Centers are often the result of dropping passes thrown to them, or from making a bad pass. Offering art that would enhance the ability to better pass or catch a basketball could therefore also enhance overall performance for anyone playing the sport of basketball.

Another hand task in basketball is in slamming a basketball through a basketball hoop (commonly known as slamdunking). When one slamdunks a basketball it is often done with force and almost always involves striking a metal basketball hoop with at least one of your hands (mostly impacting the fingers). For Forwards and Centers especially, this hand task can quickly take a toll on their fingers if their fingers are not somehow protected. The current—and only—method of enhancing this hand task is by applying basic sports tape to select finger joints. This current method and product has significant shortcoming that often result in inferior execution. For example, if one tapes parts of ones hands with the prior art, they will lose any grip capabilities in that area, which could then result in mishandling the basketball (a significant issue with Forwards and Centers). Better devices and methods of providing added protection while slamming while not sacrificing ball control would significantly impact the sport of basketball.

Other general hand task challenges that are in need of a better solution have to do with basketball players who injure, in some way, their hand. In this situation the player has to tape their hand (or wear some sort of medicated bandage), especially when injuring a finger. The result again is a decrease in ball control and limited protection. To protect the injury, most players will choose to tape the finger and try to adjust. Loss of dribbling control often results, as does loss of ball control when preparing to shoot the ball (as seen when the ball slips out of the players hand and flies up in to the air as they are shooting the ball). If a player injures herself, the player will have diminished gripping capabilities if she uses prior art to cover and protect that injury. Additionally, athletes often can stress joints beyond their limitations. Injuries can result when limbs are bent beyond the natural elastic limitations of tendons and muscles. Adhesive tape is commonly used as a propylaxis. Tapes are therefore also used to protect from injury or to protect one from further injury.

Therefore, new art which could both protect an injury as well as provide a grip enhancing element (such as PVC dots on the outer surface of the invention) could have a significant and substantial impact on the sport.

### DETAIL DESCRIPTIONS OF THE INVENTION

In general the present invention offers grip enhancing capabilities for the arm, the hand and in select areas of the arm and/or the hand. These arm and hand grip enhancers can be adhered to the skin of an individual, and thereby increase, among other things, the grip capabilities of that area. The present invention offers a grip enhancing sports tape that is designed to be adhered to an individual using an adhesive means on one side, the bottom surface, of the present invention, and comprising a gripping means, on the other side, the top surface, of the present invention.

The gripping means is centrally located along the sports tape top surface, said sports tape. Embodiments of said sports tape may be configured as one layer of fibers, or may be constructed as a first layer, or base layer, and a second layer consisting of a gripping means that is affixed or otherwise permanently bonded to the top surface of said base layer.

According to one aspect of the invention, the present invention is a sports tape having a top surface and a bottom surface. The bottom surface further comprises of an adhesive means which may touch, adhere and at times wrap around the arm or hand of an individual. The sports tape also provides a gripping means, such as, for example, a plurality of projections in the form of PVC dots, and covers between twenty percent and sixty percent of the sports tape top surface, thus creating a higher coefficient of friction than the skin of a user, on the sports tape top surface.

Moreover the gripping means overlaying between twenty and sixty percent of the sports tape top surface will have a higher coefficient of friction than the remaining sports tape top surface, which is absent the gripping means.

The gripping means permits the individual to better grip an object or device once the sports tape is adhered to the individual by the adhesive on the bottom surface. The present invention can be configured to attach directly to the skin of the human body, or attach to an item already on the human body. The gripping means can be integrally formed by, for example, embossing the sports tape top surface to create the gripping means, such as irregular depressions of, say, at least 100 micrometers in depth. The adhesive layer on the bottom surface can be coated, for example, with a pressure sensitive adhesive based on natural or synthetic elastomers, such as amorphous polyolefins.

In another preferred aspect, the present invention is comprised of, for example, a sports tape which includes an additional layer comprised of an medicated additive on the bottom surface, such that the tape can be used to, for example, protect an injury or to protect an area from being injured. The present invention would then offer the unique ability of being able to protect an injury without minimizing grip capabilities in said areas, a significant and substantial advancement to prior art, such as bandages and BAND-AID. These embodiments, as will also be discussed, may be achieved by, for example, coating a portion of the bottom surface of the embodiments with a medicated additive.

The adhesive composition of the present invention may be prepared using standard methods. For example, the adhesive composition may be prepared by uniformly mixing the raw materials by a convention mixing method. For example, the amorphous polyolefins, the medicated additive or the therapeutic additive, etc. are mixed with a mixing apparatus such as a homomixer or a planetary mixer to homogeneously dissolve or disperse the materials to obtain the liquid composition. It is then applied using standard methods, as mentioned herein.

In still another preferred aspect, embodiments can additionally benefit users to, among other things, protect an injury, and/or protect from injury, by offering a shock-absorbing member as an additional layer, or a middle layer, for embodiments of the present invention. The shock-absorbing member may comprise foams including but not limited to open-cell or closed-cell foams, such as BOLLARD foam, polyolfin foam, polyurethane foam, and the like. Additionally, the foam may possess a substantially uniform cell distribution.

Embodiments may also offer important moisture repellency by providing a moisture repellant coating on the fibers forming the sports tape, such as a synthetic resin, or by providing an adhesive means of the bottom surface that has moisture repellant capabilities, such as a porous adhesive or a water-resistant adhesive, such as a polyacrylate adhesive, or by forming the base layer of the sports tape with a moisture repellant fabric such as GORTEC. Embodiments may therefore comprise various weather-resistant and perspirant-resistant materials, forms and designs including, but not limited to, water-resistant materials or hole designs for moisture management, or combinations thereof.

In a further preferred aspect, embodiments can be constructed as a base layer fabric or may be configured as improvements to conventional dermal sports tapes designed to be adhered to the skin of a user, by creating a second layer, said second layer comprised of one panel which comprises the gripping means on the panel top surface.

Those embodiments comprising a gripping means as a panel will have formed on said panel either a plurality of projections as described herein, or a plurality of depressions as described herein. Finally, formed of said panel top surface may also be comprised of a tackifier coating or tackified leather consisting of the materials forming said panel; said tackifier coating or tackified leather being designed to provide a Shore A Durometer Coefficient of Friction of between 2.0 and 4.5; more preferably, said tackifier coating being designed to provide a Shore A Durometer Coefficient of Friction of between 2.5 and 3.5.

Said panel is then bonded or otherwise affixed to a centrally located portion of the top surface of said conventional sports tape, using heat sealing or other standard methods. In either case, the base layer or the conventional sports tape is designed to have flexible and elastic properties. Preferably, the base layer has at least ten percent stretch capabilities; more preferably, the base layer has between fifty percent and one hundred eighty percent stretch capabilities; still more preferably, the base last has at least one hundred percent stretch capabilities; even more preferably, the base layer has between one hundred percent and one hundred fifty percent stretch capabilities.

The panel is formed, however, of preferably flexible but generally not very elastic fibers. Preferably, the panel is designed to have at most about fifty percent stretch capabilities. More preferably, the panel is designed to have at most twenty percent stretch capabilities; even more preferably, the panel is designed to have at most ten percent; still even more preferably, the panel is designed to have at most five percent stretch capabilities.

These generally inelastic panels are affixed to the base layer of sports tape embodiments. More specifically, the panels overlay a centrally located portion of the base layer top surface. The centrally located panel has a smaller width dimension, compared to the base layer width. Preferably, the centrally placed panel overlay between twenty percent and sixty percent, widthwise, of the base layer top surface. Many embodiments comprise of a centrally located panel layer that has approximately the same length of the sports tape base layer.

Providing a less elastic panel layer over a centrally located portion of a generally elastic base layer offers significant and substantial results. The panel allows a user with a stable central portion top surface which may overlay his or her phalanx, while the smoother, untextured and elastic edges of the sports tape still allow a user to flex or clench a hand around the finger joints, a critically important feature and combination not offered by prior art.

If the panel overlays more than approximately sixty percent, the sports tape unnecessarily constricts finger flexibility when gripping a sports device, such as a golf club, and thus would defeat the purpose of its intended use. If the panel overlay less than twenty percent, the sports tape will provide too little grip enhanced capabilities, and far too much of the sports tape will be very elastic with over eighty percent unrestricted and creating an unstable grip platform or top surface.

Some embodiments will comprise of a gripping means that is formed on said sports tape top surface without a panel layer. Said gripping means comprises either of a plurality of projections as described herein, or a plurality of depressions, as described herein. Finally, formed of said sports tape top surface may also comprise of a tackifier coating; said tackifier coating being designed to provide a Shore A Durometer Coefficient of Friction of between 2.0 and 4.5; more preferably, said tackifier coating being designed to provide a Shore A Durometer Coefficient of Friction of between 2.5 and 3.5.

Said gripping means is then created by any standard methods, such as embossing or otherwise, onto a centrally located portion of the sports tape top surface of said conventional sports tape.

In either case, the base layer or sports tape top surface is designed to have flexible and elastic properties. Preferably, the base layer has at least ten percent stretch capabilities; more preferably, the base layer has between fifty percent and one hundred eighty percent stretch capabilities; still more preferably, the base last has at least one hundred percent stretch capabilities; even more preferably, the base layer has between one hundred percent and one hundred fifty percent stretch capabilities.

The materials forming the gripping means, however is preferably flexible but generally not very elastic. Preferably, the gripping means is designed to create less than fifty percent stretch capabilities along the centrally located portion where the gripping means resides. More preferably, the gripping means is designed to create at most twenty percent stretch capabilities along the centrally located portion where the gripping means resides along the sports tape top surface; even more preferably, the gripping means is designed to create at most ten percent stretch capabilities along the centrally located portion where the gripping means resides along the sports tape top surface; still even more preferably, the gripping means is designed to create at most five percent stretch capabilities along the centrally located portion where the gripping means resides along the sports tape top surface.

These rather inelastic gripping means are affixed to the top surface of the base layer fibers forming the sports tape. More specifically, the gripping means overlay a centrally located portion of the base layer top surface. The centrally located gripping means does not overlay the entire width of any portion of the base layer top surface; it has a smaller width dimension, compared to the base layer width. Preferably, the centrally placed gripping means overlay between twenty percent and sixty percent, widthwise, of the base layer top surface. Many embodiments, however comprise of a centrally located gripping means layer that overlays approximately the entire length of the sports tape base layer top surface.

Providing a less elastic gripping means layer over a centrally located portion of a generally elastic base layer offers significant and substantial results. The gripping means allow a user with a stable central portion top surface which may overlay his or her phalanx, while the smoother, untextured and elastic edges of the sports tape still allows a user the flex or clench a hand around the finger joints where a finger expands the most when gripping a sporting device, a critically important feature and combination not offered by prior art. If the gripping means overlays more than approximately sixty percent, the sports tape unnecessarily constricts finger flexibility when gripping a sports device, such as a golf club, and thus would defeat the purpose of its intended use. If the gripping means overlay less than twenty percent, the sports tape will provide too little grip enhanced capabilities, and far too much of the sports tape will be very elastic, over eighty percent unrestricted and creating an unstable grip platform or top surface.

Some embodiments further provide a liner material (release sheet) which is configured to cover the adhesive means and keep the adhesive from drying until the user is ready to apply the embodiment to the user's skin. Said liner material can comprise of paper, such as KRAFT PAPER, and may also be made of a flexible sheet of at least one of polyethylene film, polyurethane film and the like, and generally covers the entire bottom surface area. Said liner (or release) paper is peeled off as the embodiment is applied to the skin of a user.

In still a further preferred aspect, embodiments can be comprised as a significant improvement to specialty athletic tapes, such as Kinesiology tape and Bio-tape, by providing a high friction means on said tapes.

Embodiments of these grip enhancers can, for example, increase the overall performance in arm and/or hand task activities, by allowing an individual to better control, for example, a ball or object. Embodiments can offer grip enhancing capabilities for the arm area, in particular, for example, the wrist, forearm, elbow and bicep areas, as well as for the hand, including the fingers of the hand, and methods thereof. Additionally, embodiments can also offer unique benefits by offering the ability to better protect an injury or to better protect from injury.

The gripping means is configured to provide a higher coefficient of friction than the skin of a user offers; more preferably the gripping means is configured to provide a Shore A Durometer Coefficient of Friction of between 1.0 and 4.5; even more preferably the gripping means is configured to provide a Shore A Durometer Coefficient of Friction of between 2.0 and 4.5; even more preferably the gripping means is configured to provide a Shore A Durometer Coefficient of Friction of between 2.0 and 4.5; still even more preferably the gripping means is configured to provide a Shore A Durometer Coefficient of Friction of between 2.5 and 3.5. These specific coefficient of friction ranges are significant in that they offer the best gripping capabilities of a user, significantly higher than what the skin of a user's finger, for example, would provide, and will not create too high of a friction surface such that it can begin to damage the sporting device a user is gripping while wearing the sports tape, thus making the sports tape inoperable in various sporting activities.

The adhesive means of the present invention can comprise various types and tensile strengths of adhesives configured to ‘stick’ to an individual or to an item that is placed on an individual. Adhesives used skin contact adhesive products, for example, by ASSURED LONG LASTING ADHESIVES, or BAND-AID BANDAGES, could provide sufficient tensile strength for many embodiments of similar size as the above mentioned popular products. For larger embodiments, such as sports tapes with wider widths, adhesives used by SALONPAS patches could provide the necessary bonding capabilities. Embodiments may include, but are not limited to, pressure-sensitive adhesive designed for removable application such as acrylic based and dextrin based adhesives, and/or that are configured for conventional athletic tapes that attach to a skin region of the human body. As mentioned, the adhesive on the bottom surface may preferably be water or moisture-resistant, such as any standard used water resistant adhesives, such as a polyacrylate adhesive or polymerization reaction product of two alkyl acrylate or methacrylate ester monomers such as butyl acrylate and ethyl acrylate, with a ethylenically unsaturated carboxylic acid, a vinyl lactum and a crosslinking agent. The adhesive means layer on the bottom surface can be coated, for example, with a pressure sensitive adhesive based on natural or synthetic elastomers, such as amorphous polyolefins.

The gripping means of the present invention can comprise of, for example, various grip-enhancing materials, forms, coating and designs including, but not limited to, foams, fabrics, PVC dots, perimeter patching designs, linear and non-linear grooves, or combinations thereof. The gripping means may also include high friction surfaces, textured surfaces, a plurality of regular or irregular projections, a plurality of regular or irregular depressions, non-slip materials such as eighty grit Emory cloth for example, coatings and designs, pebbled or beaded surfaces, convex or concave bumps, striations, cross-hatches, convex linear or nonlinear lines, concave linear and non-linear lines, angled ribs, random structures (such as described in FIG. 5), convex or concave ridges, crevices, and the like. Furthermore, the gripping means could may comprise of grip-enhancing coatings, in particular coatings comprised of tackifiers or tackified leather.

The gripping means may be formed on, for example, or may be an integral part of the sports tape construction thus being permanent placed of the sports tape top surface.

The present invention may comprise of various colored and multi-colored materials, forms and designs including, but not limited to, fluorescent and reflective coating.

Some embodiments may comprise various types of materials, forms, and designs usually associated with the production of gloves, including, but not limited to, stretch materials and designs, mesh fabrics, recycled and flexible materials, cottons, polyester, rayon, spandex, fleece, synthetic leathers, rubbers, plastics, or combinations thereof. Many of these embodiments may be, for example, generally elastic, although some portions on the top surface, comprising the gripping means, may be rather non-elastic while the rest of the embodiment is elastic thus allowing the ability of the user to attach the embodiment and wrap the embodiment around part of the arm area, for example. The user wrapping, say the forearm, would then wrap the embodiment is such as manner such that the rather non-elastic portion of the embodiment is located in the area of the forearm where a ball, say a football, often touches, thereby increasing the grip capabilities when carrying the ball.

Many other embodiments are constructed with standard athletic tape materials, such as, for example, woven fabrics, non-woven fabrics, or combinations thereof.

Embodiments may also comprise various weather-resistant and perspirant-resistant materials, forms and designs including, but not limited to, water-resistant materials or hole designs for moisture management, or combinations thereof.

Embodiments would not be seen as permanent grip enhancers, which is to say that they would not generally be expected to last for more than a week or two once adhered to the individual. Embodiments could, however, be configured to last more than a few hours with active use. Additionally, the adhesive used are not structural adhesives; these structural and industrial adhesives are not intended for nor would they be successful if placed on the human body

Support Embodiments for the Arm Area

One sport where the present invention will clearly enhance performance is in the sport of football. For example, one particular unmet need that a grip-enhancing device for the arm area will satisfy is with receivers. Embodiments can offer greater consistency and enhanced performance in football activities. For example, a sports tape embodiment, in the form of a wrap, can be attached to the elbow area, and then another piece of the wrap embodiment can be attached and adhered to the bicep area. The gripping means may be in the form of a grip-enhancing design such as several grooves throughout the top surface of these embodiments. These embodiments will enhance a player's ability to better absorb impact from the ground or from a defender—without losing control of the ball. Additionally, they can minimize the possibility of the football moving once in the receivers grasp because, among other things, grip capability has been enhanced throughout the arm area. These embodiments will, of course, be used to better grip the football throughout the arm area by using these embodiments to squeeze and grasp the football on both tips, or ends, of the football. A preferred width of these embodiments generally begin around two or three centimeters, but may just as easily range from four to nine centimeters or more; additionally, the embodiments may be crafted with a blend of polyester and LYCRA spandex for added durability, flexibility and elasticity. The gripping means is centrally located along the sports tape top surface, and preferably covers between twenty and sixty percent of the sports tape top surface, or base layer top surface.

These high performance sports adhesive wrap embodiments offer enhanced overall control stability and offer a high tensile strength adhesive on the bottom surface to attach securely onto the body, and therefore assist athletes more successfully perform athletic tasks. Suitable dermal adhesives can be those used in prior art athletic tape, such as that used on MCDAVID ATHLETIC TAPE. Embodiments may be spooled, for example, perforated about every inch or so, have a liner to protect the adhesive means from drying until ready for use, and be about 75 inches in length, for example.

Embodiments also significantly improve the performance of running backs, or more generally any player that runs with a ball. One embodiment of the present invention, comprising of a sports tape with a more narrow width for example, would allow a player to significantly increase his ability to control or cradle a ball when running. This embodiment would be adhered and designed to wrap around the user's wrists to help stabilize control, thereby supplementing a grip from any glove that might be currently available and in use.

Many of these embodiments could be made of material used to manufacture standard sports tape. These flexible but not necessarily elastic embodiments may offer PVC dots to provide enhanced grip capabilities on the top surface, and an adhesive typically used in sports tape, on the bottom surface, to provide the adhesive means to attach to the wrists. The adhesive can be coated with a skin-contacting adhesive, such as, for example, from a group of acrylic based, dextrin based, or urethane based adhesives. The adhesive coating may cover the entire bottom surface, or at least part of the bottom surface (for embodiments that also offer therapeutic additives on the bottom surface for added benefits). Embodiments may have the adhesive deposited onto the bottom surface, in a continuous or discontinuous pattern rather than as an overall coating, if desired.

Embodiments may also be used as a grip enhancer around the elbow area. Specifically, for example, two embodiments could be used in combination; one could be placed just below the elbow area and the other just above the elbow area, to create the ability of literally grabbing one end of the football with the elbow. These embodiments may comprise of a rubber material forming the sports tape, much like that used to manufacture a standard sports glove. Clearly, this would create a much greater, more stable overall grip because one would now have grip enhancers to increase one's grip on both ends of the football, with a glove on the hand holding one end of the football, and the grip enhancer embodiments on the elbow area holding the other end of the football.

More specifically, an embodiment for the forearm could comprise, for example, a sports tape with a wider width, say about seven to ten centimeters. The embodiment for the forearm would, for example, include a high friction outer (or top) surface as the gripping means comprised of a pattern which could be formed on, for example, a vinyl material, and would preferably be provided on at least three centimeters along the centrally located portion of the top surface and more preferably extend throughout fifty percent of the top surface of the embodiment. The embodiment for the elbow area could comprise, for example, a sports tape with a more narrow width, say about 1.5 to three centimeters. This embodiment would include a plurality of projections on the top surface as the gripping means formed from, for example, one of a vinyl material, a rubber material, or a neoprene material. The projections would preferably be provided on at least one centimeter of a centrally located portion of the sports tape top surface and may extend about 1.5 centimeters of the top surface of the embodiment. The projections could preferably extend out less than 1/10 of a centimeter, but could range generally from ¼ of centimeter to a millimeter.

Both the forearm embodiment and the elbow embodiment are adhered directly to the elbow and forearm by placing the bottom surface of these embodiments directly to the forearm and elbow area. The bottom surface, having the adhesive means, would then adhere to the skin of a user. Preferably, the bottom surface can also be adhered to skin-contact products as well, such as bandages, dressings, BAND-AIDS, patches, and the like. The bottom surface further comprises an adhesive means, the adhesive composition containing, for example, a crystalline polymer, such as acrylic polymer.

These two embodiments provide many benefits to the user. For example, by applying these embodiments to the forearm and elbow area, the football player can now use the high friction grip enhancer on the elbow embodiment to control the football and minimize ball movement at both ends of the football (and not just at the end where the football player grabs the football with his hand), while maintaining more overall stability controlling the ball with the projections on the forearm embodiment. These embodiments can, in some respect, be considered more like a glove tape: A tape with many of the benefits offered gloves—enhanced grip capabilities as well as enhanced protection, and comprised with the same materials used to construct a performance gloves—but having a significantly different physical structure. This physical difference allows the user to select which areas of the body to target, including but not limited to the hand area. Other materials that can comprise these glove-tape embodiments that are both flexible and elastic include, but are not limited to woven materials that include natural, synthetic or blends of natural and synthetic yarns, thermoextruded or thermoset rubbery embodiments including those made from thermoplastic elastomers. Examples of synthetic yarns include nylon, polyester, and spandex (polyurethane) yarns.

Other sports, such as Rugby and Mud Football, could benefit from many of the described embodiments for many of the same reasons.

Embodiments of the present invention offer significant and substantial benefits by offering the ability to better control a ball in select areas of the arm. For example, embodiments may be placed on primarily only the forearm area, allowing the player to have significantly enhanced control over a ball, much like a glove can help a player's hand better control a ball. In addition, these forearm embodiments offer new and surprising results when used in the sport of volleyball: they allow a player to impart much more spin on the volleyball—an added feature which can revolutionize the sport. This ability to impart significant spin on the ball make it easier to control the ball upon contact, to strike a ball, to get the ball to a teammate, and to redirect the ball.

One particular embodiment comprises of a sports tape designed to overlay the entire forearm of a user. Specifically, it may generally be made of a stretch or elastic, moisture-resistant fiber, with grooves along most of the top surface. Additionally the top surface may also have a more non-elastic, moisture-resistant fiber, such as a silicone based or cured silicone materials, with convex dimples along a portion of the top surface (See FIG. 8).

The bottom surface may be coated with a latex-free adhesive means layer configured to allow the embodiment to be adhered to the forearm area of the human body. Once adhered, one would now have significantly more ball control during volleyball practice or game play by providing a higher coefficient of friction than what the skin of a user provides. For example, once the volleyball approaches the athlete, the athlete can make some contact with the volleyball using the embodiment on the forearm instead of the skin of the forearm. Using the gripping means of the embodiment, such as dimples or linear grooves along the entire top surface, the athlete could not only make a more solid contact but by quickly rotating her forearm at ball contact, the grooves would momentarily ‘grip’ the volleyball and give the volleyball more spin than what could otherwise be achievable without the present invention.

The present invention thus provides a flexible, dermal adhesive product which can employ an acrylic based adhesive to provide a desired level of aggressive adhesion to skin under moist conditions. An additional benefit of this adhesion is that it is capable of being subjected to sterilization procedures and be hypoallergenic.

Some athletes may find some discomfort when peeling off some forearm embodiments. Therefore, a thin buffer tape commonly called ‘pre-wrap’ may be applied between the skin and the adhesive overlay tape embodiments. Pre-wrap is a non-adhesive product made of a foam elastomer. It is generally applied in a double layer against the skin, after which the embodiments would be wrapped on top of the pre-wrap.

The adhesion between the adhesive layer and the bottom surface is preferably very strong. To increase the adhesion between the adhesive layer and the bottom surface, a primer may be applied to the bottom surface on which the adhesive layer is placed.

Many embodiments may be considered as significant improvements to specialty tape, such as kinesiology tape, and can possess many protective as well as skin care features. The tape can comprise a fabric which includes a weave of fibers, wherein the fibers include an elastic fiber covered by a covering material; a first end; a second end, wherein the second end is opposite the first end; and one or more rounded corners; a longitudinal cut in the fabric, wherein the longitudinal cut: passes through at least a portion of the fabric; and extends from the first end to a pre-determined distance from the second end: adhesive on a first surface of the fabric, wherein the adhesive is configured to adhere the fabric to a human body; and a release sheet on the first surface of the fabric, wherein the release sheet is configured to cover the adhesive and protect the adhesive from drying until a user is ready to apply the fabric to the human body. The improvement to this kinesiology tape would comprise of the addition of a gripping means on the outer surface a second fabric, which is then bonded to the outer surface of the specialty tape. The gripping means could comprise, for example, a textured high friction outer surface. A preferred textured surface comprises PVC dots. This significant and substantial improvement would allow the user to receive the many skin care benefits of using kinesiology tape while also being able to maintain and/or increase grip capabilities along at least part of the taped areas.

Support Embodiments for the Hand Area

Grip enhancing embodiments for the hand can certainly assist an individual more effectively perform non-sport as well as sports activities, or indeed for general use. Specific areas where embodiments more effectively increase performance include, but are not limited to:
Indoor activities where a glove is often burdensome,
Gripping daily household items like twisting off a tight lid,
Placing on just thumbs and forefingers when doing a manicure,
Gripping a hammer,
General construction work

As one can see, embodiments of the present invention have all the advantages of the prior art (such as conventional sports tapes or gloves) without the disadvantages. Moreover, embodiments are also better than gloves because they offer less waste of materials. In addition, one can use embodiments to wrap a skin contact item, such as BAND-AID.

Individuals engaged in sports activities also clearly benefit from embodiments of the present invention. One sport where grip-enhancing embodiments of the present invention will clearly enhance performance in hand tasks, is in the sport of football. For example, one particular unmet need that embodiments will satisfy will be with football quarterbacks.

One embodiment of the present invention comprises of a sports tape which is adhered to the quarterback's throwing, or dominant hand. This embodiment allows a quarterback the ability to increase performance. The embodiment may be made of a natural rubber, having projections, such as PVC dots for example, throughout its top surface, and designed to wrap around a user's finger. Because football is often played outside, embodiments are preferably made of moisture-resistant fibers or coated with a moisture repellent, such as a synthetic resin for example, as well. The benefits to the user of this embodiment would include: better overall grip and better control in holding and throwing a football, higher throwing accuracy, and less fumbles. Specifically, for example, a quarterback could wrap one or more finger joints of the throwing hand with the embodiment and thus providing a higher coefficient of friction than the skin of a user's skin, while leaving other fingers bare, without the embodiment.

More specifically, for example, say a quarterback's preferred manner of throwing a football is by placing only his ring and middle fingers over the football laces (the other fingers would not be touching the laces). The quarterback might then leave his ring and middle finger bare, but perhaps adhering the sports tape embodiment in a wrapping fashion along the other fingers. This embodiment would allow a quarterback to take into account the benefits of the laces on a football, and give a quarterback the unique ability to grasp a football over the football laces on a football, with the comfort and feel of not having a glove, while adding the support that a glove might provide, over the taped thumb and forefinger. No prior art offers this unique type of targeted grip support. This embodiment would therefore increase grip capability on select fingers, while leaving the rest of the hand uncovered and able to maintain the necessary ‘feel’ of the ball, something that prior art cannot do. The resulting stronger overall grip should make for higher completed pass accuracy, more success at throwing a spiral, and generally higher consistency and performance in ball handling and control.

Another embodiment of the present invention comprises narrow width tape which can allow a receiver to increase his overall ball grip and therefore control of a football, especially when catching a football and attempting to complete a reception. These embodiments may be adhered and wrapped around one or more joints of each of the player's ten digital segments—or one's thumbs and fingers. A high friction surface is provided as the gripping means along at least half of the top surface of the sports tape. This gripping means is preferably formed from a high friction material, such as vinyl material, a PVC (PolyVinyl Chloride) material or a latex material. The football player having adhered the embodiment on one or more joints of one or more fingers would now be better able to grip and control a football (See FIG. 5). This embodiment could also be made of a natural rubber. An alternative material formation may comprise a cotton-polyester blend. A preferred breakdown is 70 percent cotton, and 30 percent polyester. The polyester thread can be spun with the cotton yarns to produce the composite with high stretch capabilities. The adhesive means can be any pressure-sensitive adhesives classified as non-structural and adapted for use on the skin on a user.

For a receiver, this and other embodiments would also allow the player to increase receptions and decrease fumbles, by being able to better cope with added stress factors, factors which would otherwise make for an unstable overall grip.

In addition to offering greater throwing accuracy and consistency, these and other embodiments could also help minimize quarterback fumbles by adding gripping support when completing other quarterback tasks, such as when ‘pumping’ the football, scrambling from being tackled, and even when catching and passing the football, especially when in ‘shot gun’ formation.

Another sport where the present invention will meet an unmet need is in the sport of GOLF. Many embodiments can be used in conjunction with each other to uniquely solve problems that have up until now been unsolved. As aforementioned, gloves are not used on a golfer's dominant hand because of the structural limitations of this prior art. For example, a golfer may want to enhance the grip around some fingers but might want some fingers uncovered as so to maintain critical feel sensitivities. New art in the form of a new kind of kinesiology tape, for example, to support a golfer's strong hand would now allow a golfer to select which fingers she wants covered and which she wants to remain uncovered. The benefits of this significant and substantial physical difference in this new art offer the golfer a more stable overall grip, better control and enhance performance. For example, one embodiment for a golfer's strong hand comprises a finger joint embodiment, attaching to one or more joints of the dominant hand's thumb, forefinger and middle finger, leaving the ring and pinkie fingers uncovered. The ‘feel’ and coordination tasks could therefore be maintained with the ring and pinkie fingers by leaving these fingers uncovered, while enhancing the overall grip of the strong hand by covering the strong hand's thumb, forefinger and middle fingers. This embodiment therefore offers the golfer a much improved method of playing the sport of golf by using grip enhancers not just on the weak hand but also on the strong hand as well, without limiting the feel and coordination requirements in a proper golf swing. Applied to the top surface of the sports tape can comprise of a high friction surface consisting of a gripping means. Preferably, the high friction surface extends between the spaced apart lengthwise terminal edges to thereby completely cover a centrally located portion of the top surface of the tape, lengthwise. Only twenty to sixty percent of the width of the sports tape would comprise of a gripping means.

The high friction surface formed by the gripping means may comprise of a plurality of projections formed from a skid resistant material, such as neoprene, PVC, rubber or the like. The plurality of projections can form a pattern on the centrally located sports tape top surface. Preferably, the high friction surface includes interstices or spaces between projections to allow the tape to flex as needed without unnecessarily compromising the rather inelastic gripping means. Preferably, the projections have an average height of at least 100 micrometers. Other similar embodiments can have uniform heights of say, 600 micrometers, but at most three or four millimeters in height. The adhesive means on the bottom surface, may be one that is water-resistant, such as a polyacrylate adhesive.

Currently, only full-fingered gloves exist for golfers, regardless of one's preferred golf grip. One very popular grip, for example, is called the ‘interlocking grip.’ When one uses this grip, the forefinger of the golfer's weak hand is placed over her strong (dominant) hand. With this grip, clearly the role of the weak hand's forefinger has less to do with grip and more with coordination and feel on the strong hand, to more effectively control the golf swing and provide greater golf swing consistency. There is, therefore, no need to cover the weak hand's forefinger, and covering the forefinger (with a glove, for example) can actually diminish the forefinger's ability to properly feel the other hand, thereby diminishing the ability to coordinate a consistent, proper golf swing. Embodiments of the present invention offer significant improvements to gloves by allowing a golfer to self-select which fingers she wants covered and which ones she doesn't. For example, one may choose to have finger embodiments wrap all the finger joints on her weak hand, except the forefinger. These embodiments may be constructed of the same material used to make a typical golf glove, such as rubber or synthetic rubber. Alternatively, the golfer may simply prefer to simply add a finger embodiment on just the pinkie finger of her dominant hand. This idea clearly also would apply to someone gipping a golf club with the ‘overlapping’ and ‘full-fingered’ methods. Among the advantages include the ability to have enhanced overall control and golf swing stability.

Another possible outcome in the way golf is played may be in how one handles a golf putter. Current proper form is not to use a glove. If one were to watch a professional golf tournament, for example, nearly all golfers would use one glove (placed on their weak hand) that they would use to grip a club for the tee shot swing, the fairway swing and even the short, approach swings. Once the golfer was ready to use their putter and putt the golf ball, they always take their glove off, deciding to grip the putter with both hands uncovered (without any sort of grip enhancer, such as a glove). An important reason why this is done is so that the golfer could choose to keep her overall feel of the putter very high by not using a glove on either hand. With the present invention, however, the golfer can now decide to use an embodiment and wrap select parts of her hands, say the thumb and pinkie finger of the dominant hand, thereby also modestly increasing her overall grip. Even a slight increase in control of the putter can make a very significant and substantial difference in performance. These embodiments may also be constructed using a softer mesh fabric and perhaps a smoother top surface but also with a gripping means as a coating or compound, such as, for example, PVC coating, such that the top surface would have a combination smooth as well as coated surface, and would now allow a golfer to choose, if she prefers, to increase the grip she has on her club, but only modestly.

Another sport where sport tape embodiments designed for the hand would meet unmet needs would be in the sport of basketball. Individuals who play basketball have to ‘feel’ as well as control a ball to perform effectively (as mentioned previously), and although they too could significantly enhance performance in controlling a ball by using a grip enhancer, prior art forces them to choose between all feel (and therefore wear no glove) or wear a glove but then lose the ability to significantly feel the basketball with the now covered hand. These players would substantially benefit from developing a way to maintain feel while increasing grip capabilities in select areas of the hand, thereby solving this significant issue. This insoluble problem can now be solved by embodiments of the present invention

Consider a highly successful basketball player like Kobe Bryant. He has played professional basketball games, often with parts of his fingers wrapped with tape. An athlete might do this for a couple of reasons. First, conventional sports tape can be used to tighten the finger joint or finger muscle, and therefore can be wrapped to protect the hand from injury. Second, it can also be used to wrap and protect a wound from further injury. So, with Kobe's hairline fracture wound on his hand, he uses conventional sports tape at least partly to protect his wound and to keep his finger joints tight, but it also results in some loss in ability to grip the ball with the wrapped portions of his hand because of the smooth often polished tape top surface which provides a lower coefficient of friction than the skin of a user, especially when prior art sports tape get wet.

One embodiment that would help a player shoot better comprises narrow width sports tape of about 3-5 centimeters, to cover only one phalanx of a user, such as the distal phalanx (finger tip), while the rest of the hand, including the palm area, can remain uncovered. These embodiments could comprise of an adhesive means on one side (all embodiments have an adhesive on the bottom side) and non-linear grooves on the top side (surface) of the embodiment. These embodiments could be made of a moisture-resistant elastic fabric. For all basketball players, but especially for those who have trouble shooting free throw shots or shooting outside of the perimeter, the uncovered palm area helps to remind them that the basketball should rarely touch the palm area when properly shooting a basketball (if the basketball touches the palm area, then there is no shooter's gap and is therefore considered improper shooting form). Because proper dribbling form also discourages the ball touching the palm area, covering the palm area with, say a glove, would not only be relatively useless in basketball, but also a waste of resources. Embodiments would allow a player to avoid covering her palm area and instead concentrate her enhanced gripping abilities in the most important area when it comes to shooting; the fingertips. The result would of course be a better overall grip and a significantly superior alternative to the prior art of basic multisport gloves that cover the hand completely forcing the basketball player to lose much of the critical requirements of being able to feel the ball as well. This embodiment may comprise of an elastic sports tape thus allowing for greater movement and increased circulation to the area having at least 110 percent stretch capability. The embodiment would comprise 100 percent acrylic adhesive on the bottom surface, and a textured high friction on the top surface. The textured top surface can comprise, for example, bumps along at least a portion of the top surface. Other examples of textured high friction embodiments, for example, include: depressions, cross-hatching, crevices, and wavy lines.

Embodiments could also satisfy the necessary requirements in dribbling a basketball as well. According to the book “Basketball for High School Players and Coaches,” (1955) Carl Bachman describes proper fundamentals of basketball dribbling: “Certain fundamentals apply to all phases of ball handling: Looseness of finger and wrist action is important, practice spinning the ball on fingertips; a basketball should never touch the heel of the hand and seldom, if ever, touch the palm.” Sport tape embodiments for the fingertips will also find success with users desiring to develop and use proper dribbling form, especially on a user's weak-hand.

Embodiments would more generally help any player, and likely could be used, for example, by those playing the position of Guard while practicing proper dribbling on their strong-hand, as well as Forwards and Centers during actual game play for added support on their weak-hand. Although players spend most of their time dribbling a basketball with their dominant hand, most Guards especially have to spend some time dribbling and controlling a basketball with their weak hand. Because players usually have more difficulty controlling the basketball with their weak hand, usually fingertip embodiments on the weak hand, for example, would significantly improve grip and control of the basketball on the weak hand. Embodiments may be constructed, for example, of a high twist, elastic, cotton tape with a rubber-based adhesive. The fingertip embodiment widths may range from 1/21 inch to 1.5 inches, with a preferred width of about % inch.

When considering the adhesive means composition, one should consider the following: first, it should be able to contact the skin for a prolonged period of time without significantly irritating the skin; the adhesive should also be flexible enough to allow some movement of the skin.

Another method of using embodiments for enhanced general basketball play is in the form of wrapping parts of all five fingers individually, and separately covered. The palm area would therefore remain bare, or uncovered. This would give a basketball player the ability to better catch a ball because the fingers would possess grip enhancing capabilities, thus eliminating the consistent problem often found in Forwards and Centers losing control of passes. This method of adhesion also provides a player with a stronger grip on the ball when passing a ball as well as with some moisture management control, thus minimizing turnovers often caused by passers, especially in Guards. The gripping means of the embodiments may be applied by any standard methods, such as by permanently embossing crisscross ridges on a centrally located portion of the top surface of the tape and coating a pressure-sensitive adhesive means layer to adhere the tape to the skin, on the bottom surface. These embodiments therefore comprise of one backing material or base layer, and not require the bonding of a second material layer to apply the gripping means.

Embodiments designed for select portions of the hand may also allow a player to be able to apply some sort of protection on select fingers, without having to sacrifice the critical ability of being able to properly feel the basketball.

For example, one common hand task in basketball is slamming a basketball through a basketball hoop (commonly known as slamdunking). When one slamdunks a basketball it is often done with force and almost always involves striking the metal basketball hoop with at least one of your hands (mostly impacting the fingers). For Forwards and Centers especially, this hand task can quickly take a toll on their fingers if their fingers are not somehow protected. The current—and only—method of enhancing this hand task is to apply conventional athletic tape to select finger joints. This current method and products have significant shortcoming that often result in inferior execution. If one tapes part of one's hands with conventional sports tape, for example, the player will lose any grip capabilities in that area, which could easily result in mishandling a basketball. One grip-enhancing finger embodiment comprises of a sports tape finger joint embodiments for the base joints (proximal phalanx) of the hand. Again, the bottom surface would offer an adhesive means such as a hypoallergenic adhesive that is also waterproof. The top surface would offer a gripping means, such as a plurality of latex projections. Among the results would be a better device and method of providing added protection while slamming, and not having to sacrifice ball control. This embodiment may be of about 97% cotton and about 3% Nylon, thereby offering significant flexibility with at least 110% stretch capability. A secondary but still important feature is that this embodiment will help players slamdunk a basketball by helping the player to ‘palm’ the ball. This feature would provide added grip support and greater performance in slamdunking.

Other general hand task challenges that embodiments would help solve has to do with basketball players who injure, in some way, their hand. This situation is very common in basketball, where a player has to tape her hand after, say, injuring a finger. The result again is a decrease in ball control and limited protection. Embodiments allow a player to essentially ‘tape’ her finger by sticking one of these embodiments on her hand. Embodiments may also preferably include a therapeutic additive on the bottom surface, such as aloe vera for example, which could also be used for skin care reasons.

Medicated Additives include medicinal compounds, such as antiseptics, antibiotics, anesthetics, neomycil sulfate, bacitracin, and the like.

Therapeutic Additives however, include tretinoin, alpha hydroxyl acids, aloes, and other similar products that are well known.

These finger embodiments would be substantially superior because these embodiment would, for example, be able to protect a finger injury (with a therapeutic additive) while not diminishing grip, because of the gripping means on the top surface of said embodiments.

Embodiments may also be adhered to a skin-contact product, such as a bandage already on the injured area. Specifically, for example, the adhesive could be applied along the edges of the bottom surface, covering up to 50 percent of the entire bottom surface area, for example. The remaining portion of the bottom surface may then be coated with a therapeutic additive. There are, of course many other potential coating patterns which could be useful.

In general, the sport tape embodiments for a user's finger can generally be used in conjunction with any type of hand task activity and/or sports play. As discussed, they offer, among other things, an individual with the opportunity to increase overall hand task performance. Maintaining or increasing overall control, for example, can provide many benefits to a user of these, and other embodiments. Among the many benefits of the hand task enhance embodiments are that they:
Allow an individual to maintain or increase control of a ball or object
Offer the ability to grip as well as feel a ball and/or device;
Provide the unique solution for players who desire better grip capabilities only in select areas
Allow a player to protect an injury on a finger without losing significant grip capabilities—disadvantages that using prior art sports tape would create if not used in conjunction with the present invention
Give a means for players who want added protection to successfully complete certain activities, such as slamdunking a basketball, without losing grip capabilities that the prior art sports tape would create.
Provide a player with the added convenience to select which fingers to cover and which fingers to remain uncovered, simply even just to maximize overall comfort during sports play
Offer a more stabilizing overall grip of a ball or object, by conveying grip-enhancers to select locations of the hand.

Additionally, these embodiments can provide:
Improved performance in hand task execution
improve overall grip
improve stability of overall grip throughout the hands
more control
more consistency
more hand coordination by adjusting enhancers to match one particular golf swing
less ball mishandles
Be adhered to an object already on an individual, such as, but not limited to, a bandage, basic tape or wraps.
Be adhered around a wound or over a wound-contact product by offering a skin care additive
Allows ability to increase grip in only select areas only

These are among the many benefits of the present invention, and are not to be construed as limitations of the benefits nor their legal equivalents

Although the description of the present invention only discussed a few embodiments, it is understood that non-sport might benefit as well from the present invention and its legal equivalents. In addition, only some embodiments have been discussed and in no way is intended to limit all the various embodiments and other embodiments that the present invention provides, such as but not limited to, different designs, different grip configurations, different adhesives, and different combinations. These embodiments can be used by men and women, boys and girls, playing any position in any sport, as well as those whose dominant hand is the right hand or the left.

A single grip can have a variety of finishes, one portion of the exterior (top) surface can have a smooth finish, for example, and another portion can have a textured surface. The textured portion would create a coefficient of friction (grip enhancer) on the surface.

There has thus been outlined, rather broadly, the more important features of the invention in order that the detailed description of the drawings thereof that follows may be better understood, and in order that the present contribution to the art may be better appreciated. There are, of course, additional features of the invention.

In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and in being used in other ways. Also, it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

### BRIEF DESCRIPTIONS OF THE DRAWING

It is expressly understood that the following descriptions and drawing are for illustration purposes only, and in no way are intended to limit the scope of the present invention and its various embodiments. For example, the drawings are of drawings of embodiments for the left hand but can easily be created for the right hand, and can be used by men and women, boy and girls.

FIG. 1 is a drawing of a first preferred embodiment, showing the top surface.

FIG. 2 is a drawing of the first embodiment, as illustrated in FIG. 1, but in a stable coiled state.

FIG. 3 is a drawing of a second preferred embodiment of an improved sports tape, with depressions as the gripping means.

FIG. 4 is a partial side view of a third preferred embodiment with a gripping means, as a layer of ridges formed thereon, improving kinesiology tape.

FIG. 5 is a drawing of several embodiments wrapped around select areas of a hand, showing the palmar side of a user's left hand.

FIG. 6 is a partial side view of another preferred embodiment used on a wrist area, said embodiment being sterilized and hypoallergenic.

FIG. 7 is a side view of still another preferred embodiment with a gripping means.

FIG. 8 is a drawing a top angled view of still another sports tape embodiment.

FIG. 9 is a drawing of the embodiment as illustrated in FIG. 3, shown adhered to the forearm and bicep area of a user.

FIG. 10 is a drawing of the several embodiments illustrated in FIG. 5, showing the dorsal side of a user's left hand.

### DETAILED DESCRIPTION OF THE DRAWINGS

It is expressly understood that the drawings are for the purpose of illustration and description only and are not intended as a definition of the limits of the invention.

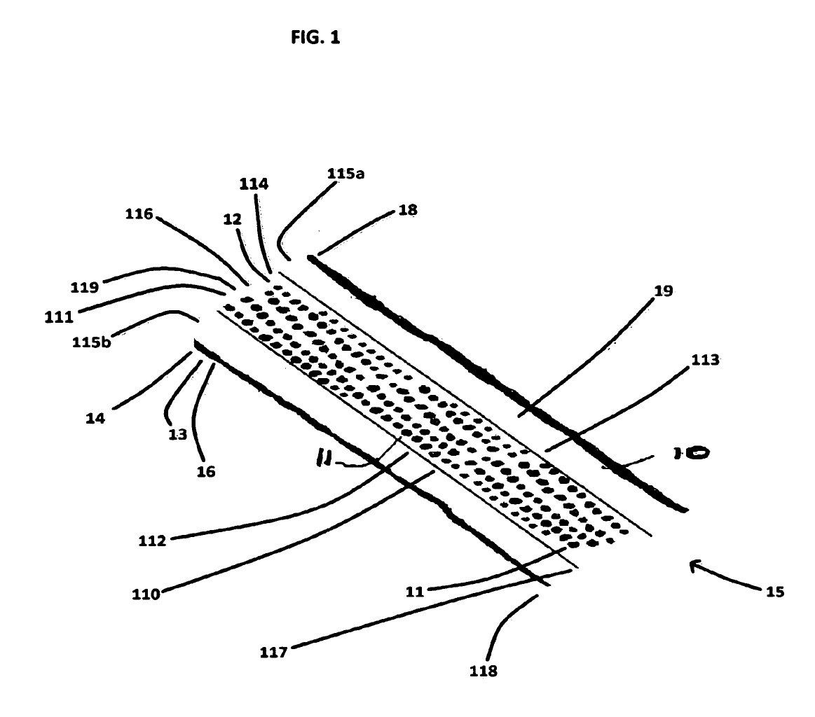

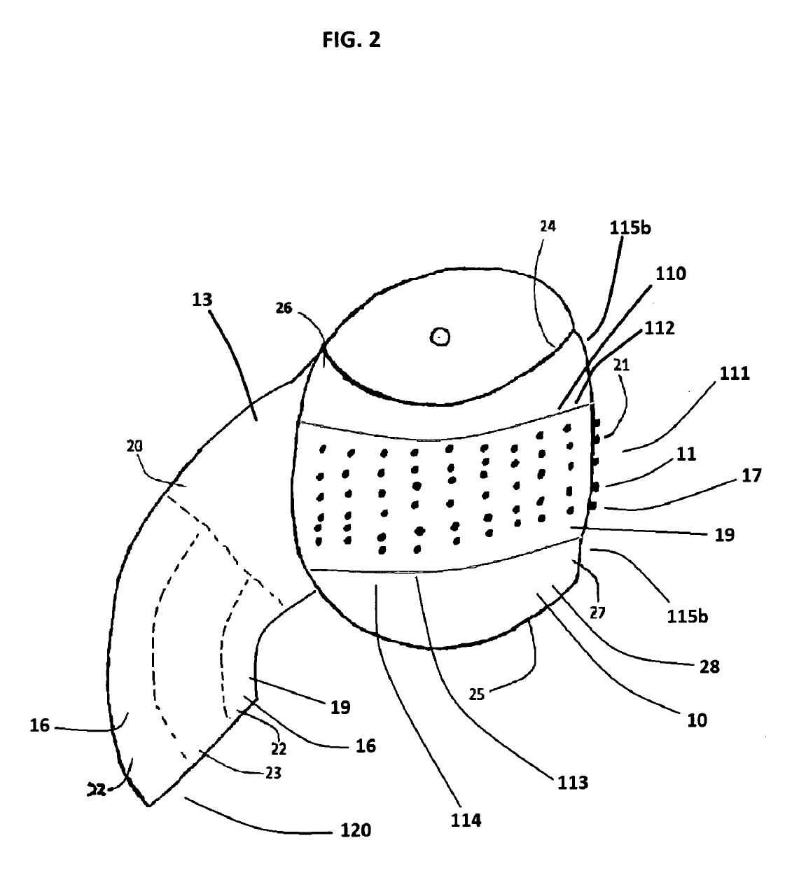

Now referring to FIG. 1 and FIG. 2, the present invention is illustrated as a drawing of a first preferred embodiment, showing both the bottom surface, and the top surface. The embodiment is a sports tape which is formed by a flexible band, twisted to show both the bottom and the top surfaces. The top surface 10 provides a gripping means 11 as defined in the specification, such as a plurality of projections, such as PVC dots 11 for example. A preferable height of the top surface plurality of projections have a height of at one hundred micrometers, preferably at least about three hundred micrometers 11, and can be imparted by any standard methods, such as, for example, by molding. The heights of the plurality of projections in this embodiment are all the same height, and are in rows 12. Other embodiments could of course offer different heights, non-uniform heights, and have a more random pattern on the top surface. The bottom surface 13 provides an adhesive means as defined in the specification and configured for attaching to a skin region of the human body. The adhesive means may be a layer comprising of a pressure sensitive adhesive 16, for example, and can be coated onto the sports tape, or by other standard methods. The adhesive means may also be, for example, an acrylic based adhesive or a hypoallergenic adhesive 22 that is waterproof 19.

The flexible band forming the sports tape embodiment 15 can comprise of a base layer formed of a fabric which includes a weave of fibers, wherein said fibers include an flexible and elastic fiber, such as cotton, covered by a covering material, such as a thin plastic; a first end 14; a second end 18, wherein the second end is opposite the first end; a longitudinal cut in the fabric, wherein the longitudinal cut: passes through at least a portion of the fabric; and extends from the first end to a pre-determined distance from the second end; wherein said base layer has two terminal edges, representing its two latitudinal edges 118, 119; the distance between said two terminal edges 118, 119 does not change throughout said sports tape length. In other words, the width of the sports tape embodiment does not change. For example, the width of the sports tape at the first end 14, 119 of the sports tape has approximately the same width as the width along the second end 18, 118 of the sports tape. In other words the distance between the first end 14 and the second end 18, remains the same throughout the sports tape.

The sports tape embodiment may comprise of a second layer 110, said second layer is affixed to a centrally located portion 111 of the base layer top surface. This second layer is formed of a flexible fiber 112 in order to have twist capabilities, but with little elasticity or stretch capabilities, such as a PVC material 113 or a latex material. Formed on the second layer is the plurality of projections 17, which provides enhanced gripping capabilities and gripping means 11, and creates a higher coefficient of friction than the skin of a user. The gripping means formed on the second layer may also comprise of a high tack coating, or tackifier compound whereby this coating provides a Shore A Durometer Coefficient of Friction of between 2.0 and 4.5. The gripping means formed on the second layer may also comprise a plurality of depressions with a depth of at least one hundred micrometers, preferably at least three hundred micrometers, and preferably not more than three millimeters in depth.

The second layer does not cover the entire top surface base layer. Centrally located on the base layer top surface 111, the second layer overlays between twenty to sixty percent 114 of the base layer top surface, leaving at least forty percent of the base layer smooth—twenty percent of each widthside 115a and 115b—and free to stretch by being unconstructed by the more rigid second layer.

The second layer 110 therefore has a smaller width 116 compared to the base layer, but has approximately the same length 117 of the base layer.

The flexible but inelastic second layer allows enhanced grip along substantially the middle portion of a finger phalanx for example, while still offering significant stretch capabilities along a substantial portion of the sports tape, allowing a user to more easily bend one's finger phalanx along the edges of said phalanx which provide the greatest stretch requirements.

The thickness of this embodiment is about one millimeter, but can be thinner (if for example the gripping means is in the form of a PVC coating) or thicker if, for example, the top surface has depressions. A preferable width for the embodiment is about 0.75 inches defining the two terminal ends, a standard athletic tape width. Similar embodiments could offer different widths. Other preferable widths might be as much as several inches. The embodiment has been cut from its coiled state and is now about six inches in length. The thickness of the adhesive layer is at least about twenty micrometers, and can be preferably range from ten to five hundred micrometers, in particular from fifteen to two hundred micrometers. Again, other materials and other adhesives can be used, such as those aforementioned, especially water-proof adhesives or by coating the fibers 10 with a moisture repellent, such as a synthetic resin 19.

This embodiment offers several benefits, especially to users needing to grip something. For example, in the sport of baseball, individuals playing the position of Pitcher have to constantly grip and throw a baseball, and the ability to grip the baseball is critical in success. Currently Pitchers do not use a glove on their dominant (throwing) hand, primarily because Pitchers also need to be able to properly ‘feel’ the ball. Wearing prior art gloves would increase grip capabilities but would significantly diminish ‘feel’ ability to the point that Pitchers currently do not wear grip enhancers on their dominant hand. This embodiment solves the dilemma faced by athletes such as Pitchers, for example, by now offering a product and a method to increase one's grip by adhering the embodiment in preferred areas of certain finger joints, while leaving other joints or the palm uncovered, or bare, therefore allowing the athlete to maintain necessary feel capabilities. For example, a Pitcher could wrap the distal phalanx of the thumb and leave the remaining finger areas unwrapped (bare). When the Pitcher now grips a baseball, his grip and overall control will be enhanced by the embodiment being wrapped around only select areas of the hand, while his overall feel of the ball will be maintained throughout the rest of his hand. It is expected that athletes playing other positions in baseball will also benefit from using embodiments on their dominant hands. In other sports, such as golf for example, embodiments may indeed be a possible substitute to the use of gloves.

FIG. 2 is a drawing of the first embodiment, as illustrated in FIG. 1, but in a coiled state. The embodiment shows a liner material (release sheet) 20 which is configured to cover the adhesive means and keep the adhesive from drying until the user is ready to apply the embodiment to the user's skin. Said liner material can comprise of paper, such as KRAFT PAPER, and could also be made of a flexible sheet of at least one of polyethylene film, polyurethane film and the like, and generally covers the entire bottom surface area of the base layer. The liner paper is peeled off as the embodiment is applied to the skin of a user. As previously mentioned, this preferred embodiment comprises of a high friction surface created by the gripping means, in the form of PVC dots 21 that is affixed to the top surface base layer. Additionally, the base layer bottom surface has a portion of the liner material stripped away to show the adhesive layer 22 which also has a therapeutic additive 23 to the bottom surface 13, such as trentinoin 23, alpha hydroxyl acids, aloe vera or other products that are well known. It is therefore beneficial that some embodiments be sterilized 28 using standard techniques. Especially for embodiments offering the above mentioned additives 23, users would find these features beneficial not only in attaching embodiments directly onto the skin of said users, but also onto a bandage already on an injured skin area of the user. The adhesive means layer 22 of this embodiment is therefore separated from the therapeutic additive compound on the bottom surface; other embodiments may combine these compounds using standard methods.

The gripping means, in the form of a plurality of projections, extends from the first terminal end 24 to the second terminal end 25 of the top surface second layer 110, leaving about forty percent smooth on the edge of each terminal edge 26 and 27, for easier user application and added elasticity. The total length of the embodiment is about seventy inches 120, and is constructed and spooled using standard tape construction methods.

Adhesives and skin care additives of the present invention and may be applied by any suitable method, such as, for example, solvent coating in a continuous or discontinuous method, air knife coating, rod coating, electrostatic coating, slide hopper coating, extrusion coating, blade coating, and slide coating.

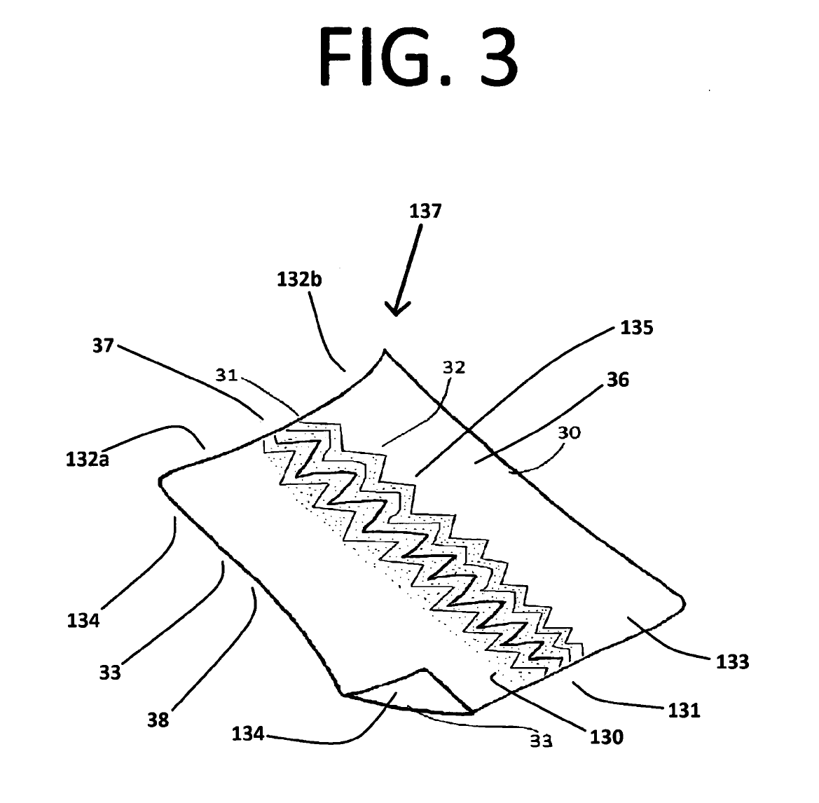

FIG. 3 is a drawing of a second preferred embodiment 137 of a sports tape having a top surface and a bottom surface, with depressions 32 as the gripping means 135 along the sports tape top surface. A preferred embodiment material 36 comprises of cotton warp yarns combined with textured polyester, such as polyamide for added stretch capabilities. Preferably, the embodiment has a length and width that is about four inches 30 by about two inches 31, respectively, but can also be designed to be wide and long enough to create a sheath on the forearm (to cover an entire forearm) without having to wind the embodiment around the forearm in an abutting fashion, say, for example a 4×7 inch embodiment. The depressions are in the form of non-linear lines 32 with depths of at least about one hundred micrometers. The bottom surface 33 is coated with an adhesive means 134, such as an acrylic pressure-sensitive adhesive 134 for example, by, say, reverse roll application on a spread line. The acrylic adhesive can then be polymerized in an emulsion using the monomers ethylhexyl acrylate, methyl acrylate, and the like, thereby providing a moisture resistant adhesive.

The gripping means 135 formed by the plurality of depressions are centrally located 37 along the top surface of the sports tape. Additionally the plurality of depressions 32 may further comprise of a high tack coating 130, or tackifier compound whereby this coating provides a Shore A Durometer Coefficient of Friction of between 2.0 and 4.5.

The gripping means does not cover the entire sport tape top surface. Centrally located on the sports tape top surface, the gripping means overlays between twenty to sixty percent 131 of the sports tape top surface, leaving at least forty percent of the top surface smooth and untextured—say about twenty percent of each widthside 132a and 132b—and free to stretch by being unconstructed by the more rigid gripping means and high tack coating.

The sports tape also comprises of a moisture resistant coating 133, such as SCOTCH GUARD.

This embodiment may be suitable for attaching to the human body by wrapping said embodiment over a skin contact product already on the user's body. For example, an athlete such as a football running back would find the embodiment very useful when attaching the embodiment on his forearm. Although the embodiment would clearly significantly and substantially increase the player's ability to grip and control a football, some discomfort might occur when the time came to remove the embodiment from the forearm, primarily because of the rather sensitive hairs on the forearm. Some using the embodiment may therefore elect to ‘pre-wrap’ using one of the many pre-wrap products on the market. The embodiment would then be attached primarily to the pre-wrap and only minimally to the skin of the user. Embodiments would therefore offer the ability to attach to the human body either directly on the skin, or by attaching to skin-contact products such as ‘pre-wraps,’ bandages, BAND-AID, and the like. The embodiment is also preferably hypoallergenic 38. Additionally, the adhesive article may also comprise a release sheet as well (as that described and as constructed in FIG. 2, for example).

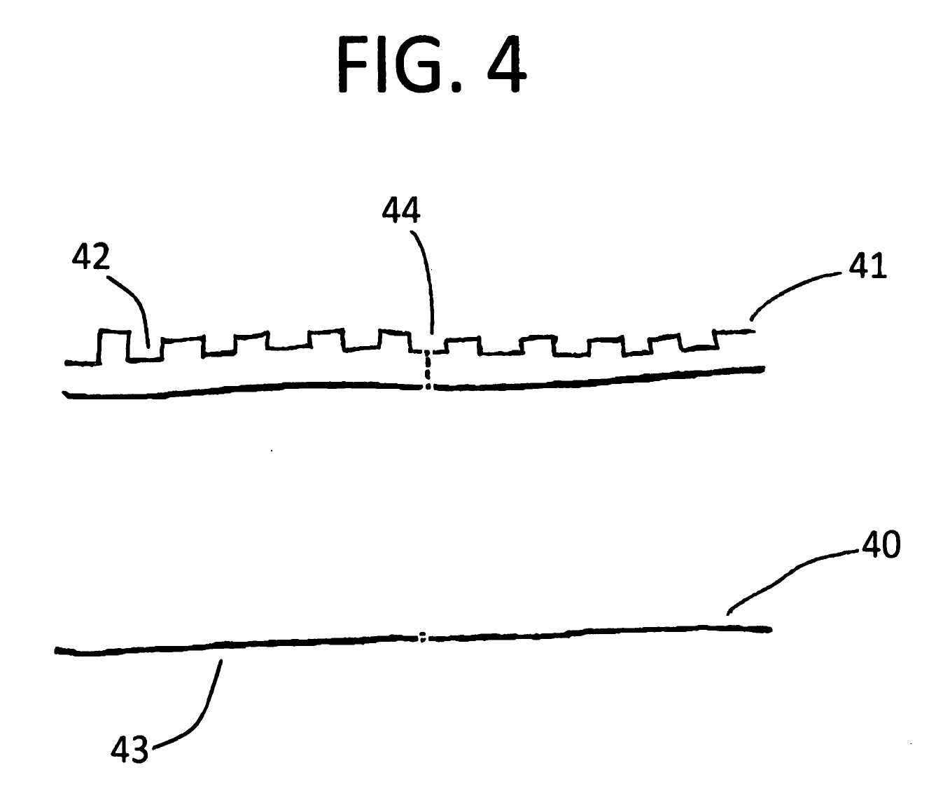

FIG. 4 is a partial side view of an embodiment showing a gripping means layer on the top surface, comprised of ridges. The embodiment comprises of a sports tape 40 formed preferably from materials typically used to configure athletic tape, such as, for example, kinesiology tape 40. For example, the sports tape—the first layer or substrate—is formed from a fabric wherein the fabric includes a weave of fibers, wherein the fibers include an elastic fiber covered by a covering material. A preferred covering material would comprise one hundred percent cotton. A second layer, comprised of, for example, a plastic material 41 would have a top surface comprising the gripping means area formed by a plurality of depressions, such as, for example, ridges 42. A preferred depth of the depressions would be such that the gap formed by the depressions would allow for some movement of the newly formed top surface edges thereby increasing the grip capabilities of the user. This gripping means has a preferred depth beginning about six hundred micrometers 42, and can be imparted by, for example, molding or standard mechanical treatments. The grip enhancing surface created by the gripping means will provide an effective coefficient of friction effective, preferably of at least about a Shore A Durometer of three but not more than 4.5. The second layer, the fiber comprised of a plastic material 41, is preferably at least about 1.5 millimeters thick and would be bonded to, and become the top surface of the cotton fiber (i.e., the kinesiology tape), by any standard method such as, for example, cementing or hot melt gluing. The new top surface now comprising a gripping means on kinesiology tape would therefore create a new kind of kinesiology tape. The adhesive on the bottom surface 43 would preferably be water or moisture-resistant, such as any standard used water resistant adhesives, such as a polyacrylate adhesive or polymerization reaction product of two alkyl acrylate or methacrylate ester monomers such as butyl acrylate and ethyl acrylate, with a ethylenically unsaturated carboxylic acid, a vinyl lactum and a crosslinking agent.

Adhesives commonly used in Kinesiology tape, such as KINESIO TEX-TAPE would of course also be suitable for the embodiment. The embodiment is preferably perforated approximately every two inches 44 or so. The perforation is configured to permit a user to easily detach the embodiment from the rest of the tape. The first and the second layers are of the same width and length, such that the gripping means will comprise essentially the entire top surface of the new sports tape. A preferred length is fifty inches and a preferred width is between two and four inches, such as about four inches 40.

Those who enjoy skateboarding would be among those benefiting from this embodiment. By attaching the embodiment onto select areas of the hand, the user would be able to better grip the skateboard when performing ‘jumps’ and other sophisticated maneuvers on the skateboard. The ridges will uniquely allow the skateboarder to maintain a stronger overall grip, while the water-resistant adhesive would ensure that the embodiment would remain attach to the skin during outdoor activities. This embodiment additionally provides a significant and substantial improvement to kinesiology tape by offering the grip-enhancing layer to said products, such as, for example, ridges and other high friction surfaces. The sports tape 40 can also consist of a pressure sensitive adhesive coated onto the backing material, such as paper or cloth. The second layer 41 may also comprise a thicker, sturdier material, say a cured silicone material, with a coarse textured surface, and bonded to the sports tape 40, creating a gripping means on a now improved sports tape.

The embodiment may also comprise of adhesives generally used for kinesiology tape along with structures and materials used to create standard Kinesiology tape, such as those comprising the very successful KINESIO TEX TAPE. Added to these tapes would be the grip-enhancer on the top surfaces. Users of kinesio tape would certainly benefit from this new type of kinesio tape, especially when the tape is applied to the hand and arm areas, where prior art kinesio tape offers a relatively low coefficient of friction. Additionally, these embodiments may comprise a liner sheet, as described in FIG. 2. Embodiments may also significantly improve other specialty tapes, such as that disclosed in U.S. Pat. No. 6,077,589 to De Carvalho, by imparting a gripping means, as aforementioned, to the De Carvalho invention.

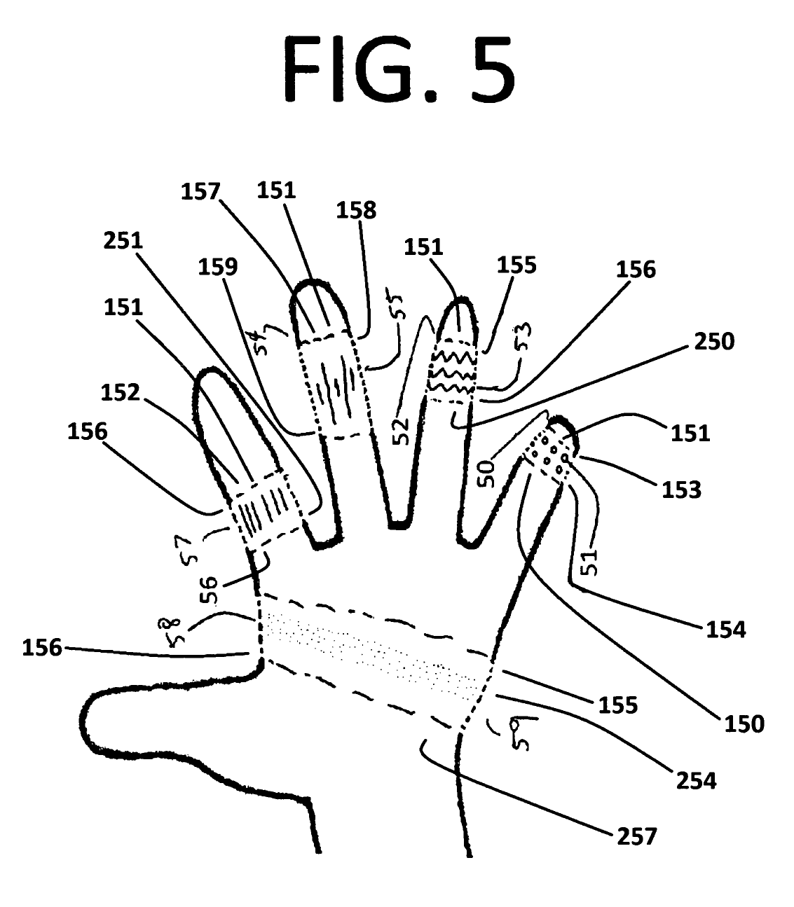

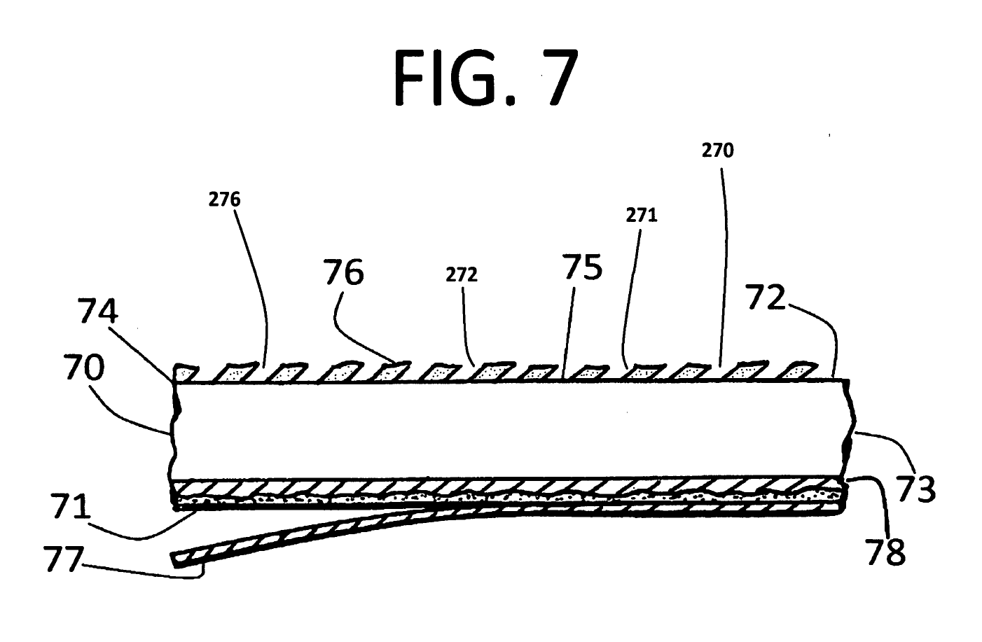

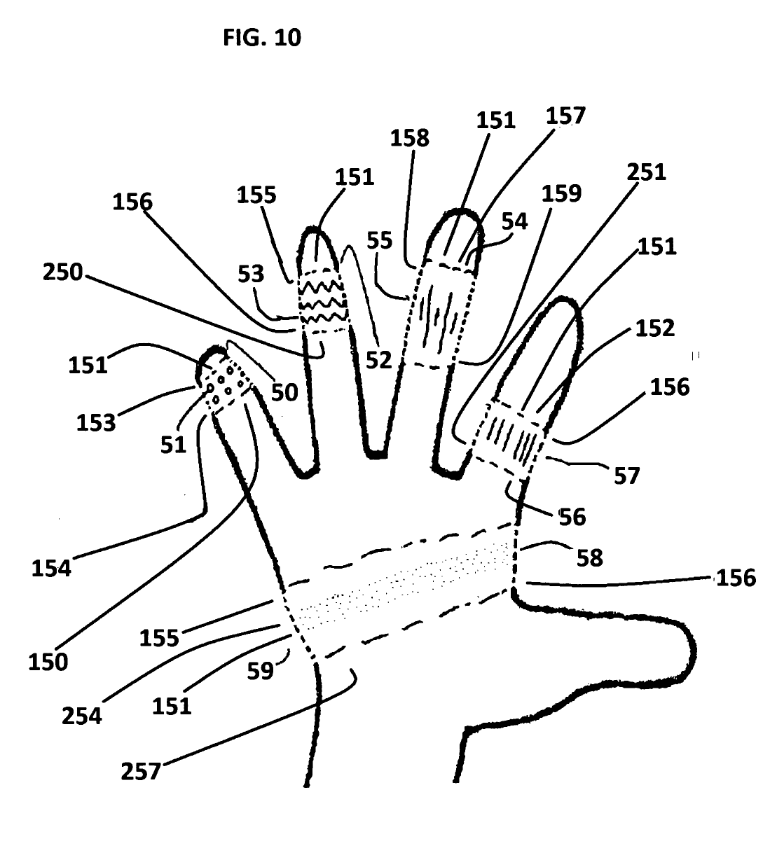

FIG. 5 and FIG. 10 are drawings of several views of embodiments wrapped around select areas of a hand, illustrating how embodiments may simultaneously cover the palmar portions (FIG. 5) and dorsal portions (FIG. 7) of a user.

These embodiments have, of course, an adhesive means on the bottom surface of each embodiment, and said adhesive means can comprise of any of the aforementioned adhesives designed to adhere the embodiments to the human skin or to a skin-contact product. These embodiments can be water resistant by coating the fibers forming the sports tape with a moisture repellant, for example, a synthetic resin 151. Additionally, these embodiments are flexible, dermal adhesive products for conformable application.

The embodiment covering the distal phalange of the pinkie finger comprises an adhesive means 50 on the bottom surface adhered to the pinkie finger and is about one centimeter wide. It has a gripping means 51 in the form of a plurality of depressions 51, such as horizontal circular grooves 51, of about three hundred micrometers in depth throughout the central top surface area of the sports tape, covering approximately fifty percent of the top surface. The remaining approximately fifty percent, about twenty-five percent on one side 153 and about twenty-five percent on the other side 154 adjacent the gripping means, are therefore uncovered by the gripping means thereby allowing the edges of the embodiment to stretch more freely than the portion textured with the more rigid gripping means. The embodiment is comprised of a natural latex material forming the gripping means 51, with a NYLON mesh fibers forming the sports tape 150 to have significant elastic properties of between about one hundred and ten percent and one hundred and eighty percent 150.

The embodiment covering a portion of the ring finger has an adhesive means 52 that is typically used in standard sports tape configurations sports tape configurations along the bottom surface, and has a gripping means 53 comprised of a plurality of depression 53, such as non-linear gripping grooves 53 of about two hundred micrometers in height, throughout forty percent of the central the top surface area of the embodiment, being beveled for example, along the edges for easier application as well as for added flexibility and elasticity. The remaining approximately sixty percent, about thirty percent on one side 155 and about thirty percent on the other side 156 adjacent the gripping means, are therefore uncovered by the gripping means thereby allowing the edges of the embodiment to stretch more freely than the portion textured with the more rigid gripping means. The fibers forming this embodiment comprise of a synthetic latex material designed to provide significant elastic properties of between one hundred and ten percent and one hundred and twenty percent 250, and is about 1.5 centimeters in width.

The embodiment covering a portion of the middle finger has adhesive means 54 used with skin-contact products, such as, BAND-AID BANDAGES, along the bottom surface of the embodiment, and offers a gripping means 55, such as non-uniform vertical grooves 55 of about % millimeter in depth throughout most of the central top surface portion of the embodiment. The material forming this particular embodiment comprises a rubber material 157 to have significant elastic properties, of about one hundred and ten percent 157, for example, or more, and is about two centimeters in width. The gripping means is provided along sixty percent of the top surface area, leaving the thirty percent closest to its two terminal edges 158, 159, smooth and untextured for easier application and added flexibility. The rubber construction provides glove-like properties and benefits aforementioned, while also enjoying the benefits of tape configurations.

The embodiment covering a portion of the forefinger offers an adhesive means 56 as used in ASSURED LONG LASTING ADHESIVE BANDAGES 56, and provides a gripping means 57 comprised of a plurality of depressions 57, such as vertical grooves 57, for example. This embodiment is made of standard athletic tape construction, such as a cloth encased by a thin, flexible plastic layer 152, such as polyethylene to have significant elastic properties, of about between fifty percent 152 and one hundred percent stretch capability, and is 1.5 centimeters wide. The gripping means is provided along fifty percent of the top surface area, leaving the thirty percent closest to its two terminal edges 156, 251, smooth and untextured for easier application and added flexibility.

There are several widths that the present invention may offer. Embodiments that target the hand may, for example, comprise a preferred width starting at about ½ or one centimeter, but may be as wide as two centimeters—wide enough to essentially cover an entire finger area such as a distal phalange, without having to use multiple layers in an abutting fashion. Additionally, adhesive article embodiments and tape embodiment may have perforations, such as that described in FIG. 4, for example.

Additionally, a portion of the palm area is wrapped with another embodiment 58. Athletes such as football receives may use embodiments in this fashion to better catch or otherwise control a football. This particular embodiment is preferably comprised of a cotton fiber 58, and has a one hundred percent acrylic adhesive means 257 that is latex-free. The embodiment can be configured to last a few days adhered to the skin, beneficial especially if the intended use is at least partly for skin care reasons, such as, for example, if the user would like to maintain tightness in certain muscles. The gripping means 59 comprises of a coating that provides high tack capabilities, such as a PVC coating 59, for example, or other tackifiers that are configured to provide a Shore A Durometer Coefficient of Friction of between 2.0 and 4.5, such as about 3.0, for example. The gripping means 59 is provided along sixty percent of the top surface area 254, leaving the thirty percent closest to its two terminal edges 155, 156, smooth and untextured with a gripping means, for easier application and added flexibility. A preferred width of the embodiment is about ½ inch for example 58 and more generally preferably vary in width, from ⅙ inch to 2.5 inches. Furthermore, the embodiment can be porous and breathable, and can additionally be used to support and stabilize injuries to joints, bones and muscles. This embodiment can alternatively be made of a SPANDEX material, thus providing stretchy properties, of between one hundred and one hundred and eighty percent. Additionally, this and the other embodiments in FIG. 5 can have a paper release sheet (as described in FIG. 2), may be perforated every three inches, and the gripping means may comprise of a plurality of projections (similar to that described in FIG. 2). In still another alternative but similar embodiment, the gripping means may comprise of a plurality of projections such as PVC beads of irregular heights, having a preferred minimum height of about three hundred micrometers, and the material may comprise of cloth. The adhesive and PVC beads could be transferred to the cloth by roller transfer coating or spraying, by stamping or by other standard methods. A preferred thickness of the adhesives may generally be about 0.1 mm after application, applied on the entire bottom surface of the embodiment.

The skin-contact adhesive means may also comprise of a rubber based adhesive, a porous adhesive or non-latex based synthetic adhesive, depending on if the embodiments will be for use in underwater activities and provided that they are hypoallergenic.

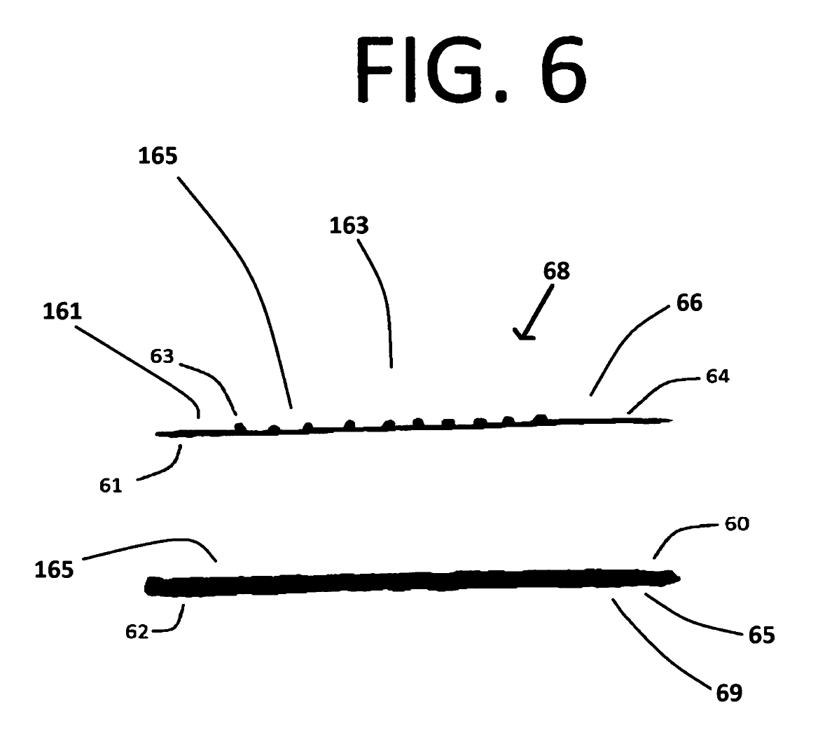

FIG. 6 is a partial front side view of another embodiment used on a wrist or forearm area wound, said embodiment being sterilized 66 and hypoallergenic 65. Sterilization may be achieved by exposure to a sterilization gas such as ethylene oxide or by radiation using actinic or electron beam radiation. In one standard method, for example, the embodiment can be subjected to sterilization by contact for up to two hours with ethylene oxide at up to one hundred and eighty degrees F. The embodiment is comprised of a stretchable material base layer 60, or first layer, of at least a one hundred percent stretch capability 60 such as a non-woven polyester-material, providing lightweight compression without causing significant constriction 60. The thickness of the tape is about 0.5 millimeters, but can generally range up to about three millimeters.

The gripping means 63 in this embodiment is in the form of a plurality of projections 63, preferably comprising of a second layer 68 formed of a more rigid plastic layer 61 which is embossed to produce a high friction surface, such as crisscross lines or a series of beaded projections. The more rigid plastic layer is preferably flexible but not as elastic as the base layer 60 and is about one millimeter thick but may preferably range, for example, from one to three millimeters thick. The plastic layer may comprise any standard flexible plastic support materials, such as, for example, a cellulose acetate, cellulose triacetate, and the like, or a polyester such as polyethylene terephthalate, or a polyolefin such as polypropylene. The embossed surface of the second layer 68 is then permanently bonded to the sports tape 60 thus creating a high friction surface on the top surface of the sports tape. The embossed layer is preferably bonded by lamination method or other standard methods, such as cementing or ethylene copolymers. Finally, the adhesive means comprised of an adhesive layer is applied to the bottom surface of the tape 62. The dermal adhesive layer 69 may be preferably at least about one hundred micrometers thick, and preferably comprises a pressure-sensitive rubber based adhesive, such as a butyl-based adhesive. Any standard method of applying the adhesive can be used, such as spraying. The fibers forming the sports tape are coated with a moisture repellant 165.

Alternatively, the tape embodiment may be assembled using standard calendaring techniques. For example, the tape and the plastic layer may be individually supplied to a calendaring operation to provide a laminar structure of these components. An adhesive layer 69 may then be coated onto the bottom surface of the tape 62 by known coating techniques. The top surface of this embodiment comprises of PVC dots 63 throughout most of the top surface, preferably about sixty percent, but can certainly extend between twenty percent and sixty percent along primarily the central portion 163 of the sports tape top surface. The top surface of the embodiment also, therefore comprises a low coefficient of friction surface 64, representing about fifteen percent of the top surface adjacent one side 64 the gripping means, with the other side of the sports tape top surface adjacent the gripping means representing the remaining fifteen percent 161. These textured finishes include surfaces having features protruding—having a height—greater than about two hundred micrometers, and can be imparted by methods such as stamping.

This embodiment offers enhanced stability with performance adhesive fabrics, and high tensile strength adhesives thus allowing the athlete to enjoy the skin care values of tape with increased gripping abilities, and therefore a higher coefficient of friction than what the skin of a user provides. There are several widths that the present invention may be. Embodiments that target the arm, for example, may have a preferred width starting at about 3 or 4 centimeters, but may have width ranges from nine to fifteen centimeters—wide enough to essentially cover an entire wrist, elbow, or even forearm area of a user. The embodiment is therefore a flexible, dermal adhesive product for conformable topical application to a human skin region, either directly to the skin, or over a skin-contact product such as a pre-wrap.

FIG. 7 is a drawing of the side view of another sports tape embodiment. Embodiments may additionally also contain a shock-absorbing member, such as a foam layer, a padded layer or a plurality of tiny cushions. For example, this embodiment for the application on a human skin region comprises a cloth material contained substantially within a plastic foam layer 72, such as, polyethylene; an adhesive means 71 in the form of a layer of adhesive 71 adhered to the bottom surface of said cloth and foam, and a gripping means 76 formed integrally and therefore permanently formed on the top surface material 72 of the sports tape, thus creating a higher coefficient of friction than what the skin of a user would otherwise provide. Other embodiments may have only the gripping means 76, separately placed on the top surface of the embodiment. The embodiment may alternatively also have a foam 70, encased in flexible material, say plastic, forming the flexible band. The shock-absorbing member comprised of foam, can be provided by heating a mixture of said plastic and a chemical blowing agent. The foam preferably possesses a substantially uniform cell distribution 73. The embodiment may be constructed by standard methods, such as coextruding a polymeric melt comprising a plastic layer of foam and a thin plastic material, such as polyethylene particles, onto a cloth material so that said foam is applied first to said cloth. The adhesive means coating 71 can then be applied to the bottom surface area by calendaring, for example. A preferred length of the embodiment is about nine inches 74; a preferred width is four inches 75. The top surface comprises a gripping means in the form of a plurality of projections 76, which is embossed creating the plurality of projections 76. These embossed shapes may be spherical, cylindrical, or elongated 76 and may be individually separated 270 or interconnected, thus creating a higher coefficient of friction than what the skin of a user would otherwise provide. These projections are about one millimeter in height 76, with an average radius of at least about three micrometers. The projections are evenly spaced, and preferably extend throughout the entire centrally located portion of the sports tape top surface area, lengthwise 74 but not widthwise 75, leaving at least forty percent of the sports tape top surface without any gripping mean overlaying it. The embodiment also shows a release sheet 77, which can be structured as aforementioned. The embodiment can also be perforated, say every two inches in length thus sealing the foam at the perforations, if preferred.

Additionally, the sports tape comprises of a second gripping means 271 in the form a grip enhancing coating. This second gripping means is formed of a high tack coating 271, such as a PVC coating, a latex coating 272 or a neoprene coating.

The second gripping means 271 is coated over the plurality of projections 76, or the first gripping means 76, and may comprise of a high tack coating, or tackifier compound whereby this coating provides a Shore A Durometer Coefficient of Friction of between 2.0 and 4.5. more preferably, the tackifier coating provides a Shore A Durometer Coefficient of Friction of at least 2.0; even more preferably with a Shore A Durometer Coefficient of Friction of between 2.5 and 3.5 271.

Neither the first gripping means nor the second gripping means cover the entire top surface sports tape. Centrally located on the sports tape top surface, the gripping means overlays between thirty to sixty percent of the sports tape top surface, leaving at least forty percent of the sports tape top surface smooth on each side widthwise, and free to stretch by being unconstructed by the more rigid second layer.

The first gripping means and the second gripping means may be designed to overlay approximately the entire length of the sports tape top surface, but the gripping means does cover more than sixty percent of the width of the sports tape.

The flexible but inelastic second gripping means, in the form of a coating layer such as a tackifier, allows an enhanced grip portion on a finger phalanx for example, while still offering significant stretch capabilities along a substantial portion of the sports tape, allowing a user to more easily bend one's finger phalanx along the edges of said phalanx which provide the greatest stretch requirements. This embodiment configuration, for example, is designed to wrap around a digital segment phalanx, such as a thumb proximal phalanx.

Once adhered to a user's thumb segment, for example, the gripping means location will overlay the user's palmar and dorsal thumb surfaces, leaving the tape portions nearest a user's two finger joints without any gripping means due to the untextured top surface portions. This significantly unique top surface configuration will thus provide a user increased gripping capabilities along generally the middle portions of a user's digital phalanx while simultaneously allowing necessary stretch capabilities nearest a user's finger joints, critically important when flexing a finger or clenching a hand when gripping a sporting device, such as a golf club.

Again, other materials and other adhesives can be used, such as those aforementioned, especially water-proof adhesives or by coating the fibers with a moisture repellent 276, such as a synthetic resin 276 such as SCOTCH GUARD.

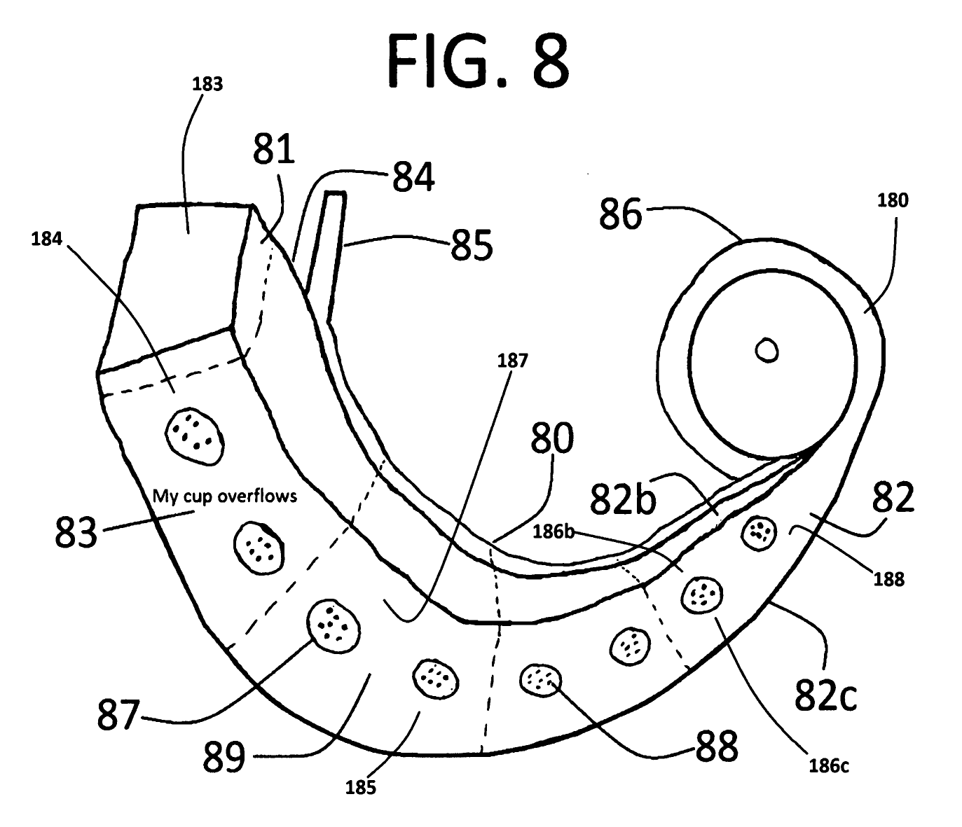

FIG. 8 is a drawing of a top angled view of another sports tape embodiment. The embodiment is a sports tape comprising a flexible band having an open-cell foam plastic core. The length of the embodiment is about seventy inches and is perforated every six centimeters 80. The flexible band may be made of a generally inelastic polyvinyl chloride foam plastic creating the open-cell shock-absorbing member 81, but may also be made of polyurethane foam padding or other padding. The foam core may have a thickness of up to two millimeters 183, preferably about one or two millimeters 183 after curing. The thickness of similar embodiments may vary depending on several factors, such as for example, the user preference. In other words, embodiments may be configured to absorb more or less by the thickness of the shock absorbing member. The embodiment thus creates a cushioning action to, for example, protect an injury on the natatory ligament area of the palm. Embodiments may therefore also be sterilized 180 and hypoallergenic 181, by methods aforementioned or other standard methods. The sports tape top surface 82 is printed as by silk screening, to identify a trademark, a slogan or a passage 83. The sports tape may be constructed, for example, by producing a molten plastic mixture of the desired composition which incorporates a foaming or blowing agent is cast in film form on a sheet of carrier paper. As the molten plastic film rises due to the foaming action, it forms a core having myriad internal cells mainly in interconnected open formation. In the course of cooling and curing, the opposing faces of the core which are exposed to the atmosphere, harden. After the cast foam plastic is fully cured on the paper carrier sheet, the bottom surface of the tape is coated with a layer of a pressure-sensitive skin adhesive 84 used on, say SALON PAS skin contact products 84, for example. The adhesive means layer 84 may be any adhesive composition aforementioned 84 of course, and has a release sheet 85 covering the adhesive layer, made of a laminate of a paper sheet and a thin plastic film. The resultant material is removed from the carrier sheet and can be fed into a slitter or other mechanism where it can be cut into individual tapes. These tapes can then be coiled into rolls 86 which are suitably packaged or mounted on a dispenser reel. The top surface of the embodiment 82 comprises of a gripping means 87 centrally located along the sports tape top surface. Specifically, the gripping means comprises a plurality of beads 88 comprised of the material of the top surface thereby being formed integral to, and permanently on, the sports tape construction. The gripping means 87 does not extend throughout more than about sixty percent of the entire centrally located portion of the sports tape top surface 184, though it does cover a substantial portion of it. This embodiment has the beads—representing the gripping means—located within a circle pattern 185 on the top surface. There is a relatively smoother space, providing a lower coefficient of friction than the portion covered by the gripping means, between the edge of each circle 186b, 186c and the terminal edges 82b, 82c, representing at least forty percent of the total sports tape top surface. In other words, the combined distance between the edge of each circle most adjacent 186b, 186c to a terminal edge 82b, 82c, and a terminal edge of the sports tape top surface 82b, 82c, respectively, comprises at least forty percent of the entire sports tape top surface.

There is also a smooth or untextured space between the circles 89 which does not have a gripping means as discussed. In other words, the high friction textured area on the sports tape top surface is concentrated to the region comprising the circles on said top surface. In general these circles may be spaced at constant distances from each other 89, said constant distance depending on several factors, such as for example, the preferred font size of the passage 83, which are generally located somewhere between the circles. This particular embodiment has about one centimeter of spacing 89 between each circle, are centrally located along the sports tape top surface 184, and are formed at a constant interval, thus creating a grip enhancing pattern throughout centrally located portion of the seventy inch sports tape embodiment top surface, leaving the remaining portions 187, 188 unconstructed by the more rigid gripping means. A preferred space range is from ⅛ centimeter to about three centimeters, for example. The spacing between the edge of the circle 186b, 186c and each side terminal edge of the tape 82b and 82c is about one centimeter, on either side. For example, this particular tape embodiment is approximately four centimeters in width, or more broadly, the distance between the sports tape two terminal edges 82b, 82c. The circles would then all have a midpoint essentially two centimeters from the width terminal edges of the tape 82b and 82c. The circles, which consist of the beaded sections of the embodiment, have a radius of about one centimeter. The top surface exclusive of the circles, would thus not possess the grip enhancing means; this would include the distance between the beginning of the circles and the two side edges of the tape 82b and 82c.

One benefit of this design pattern is that it could be designed to wrapped on and around, say the middle phalange of the forefinger, whereby the circular patterns reside along the dorsal and palmar section of said joint, while the smooth area of the top of the tape, the space between the circles, would reside on the sides of the forefinger. This would maximize flexibility and movement on the side of the forefinger while maximizing grip on the dorsal and palmar sections. Furthermore, the protective layer would be throughout the embodiment thereby protecting the entire middle phalange of the forefinger.

The embodiment as described in FIG. 8 may of course be constructed of different materials. For example the circles may comprise of a coarse material, say an Eighty Grit Emory Cloth. The one centimeter radius Emory cloth would then be affixed to the top surface of the sports tape, say MCDAVID ATHLETIC TAPE, by standard bonding methods, some aforementioned, such as hot melting or cementing, for example. Some of these embodiments may preferably be created as, say, a rather non-elastic silicone cured composition which can remain conformable but offering some added rigidity and additional moisture management properties which would also help the user with anti-slip improvements on the arm. The process may, for example, be completed by creating a silicone composition layer—or silicone sheet—of about two millimeters or greater in thickness. The silicone layer having a textured surface formed thereon, such as, for example, silicone ridges. The silicone layer would then be bonded to the sports tape. Alternatively, elastic embodiments may be coated with a gripping means on the top surface that creates a high coefficient of friction, preferably with a Shore A Durometer Coefficient of Friction of between 2.0 and 4.5, more preferably with a Shore A Durometer Coefficient of Friction of between 2.5 and 3.5.

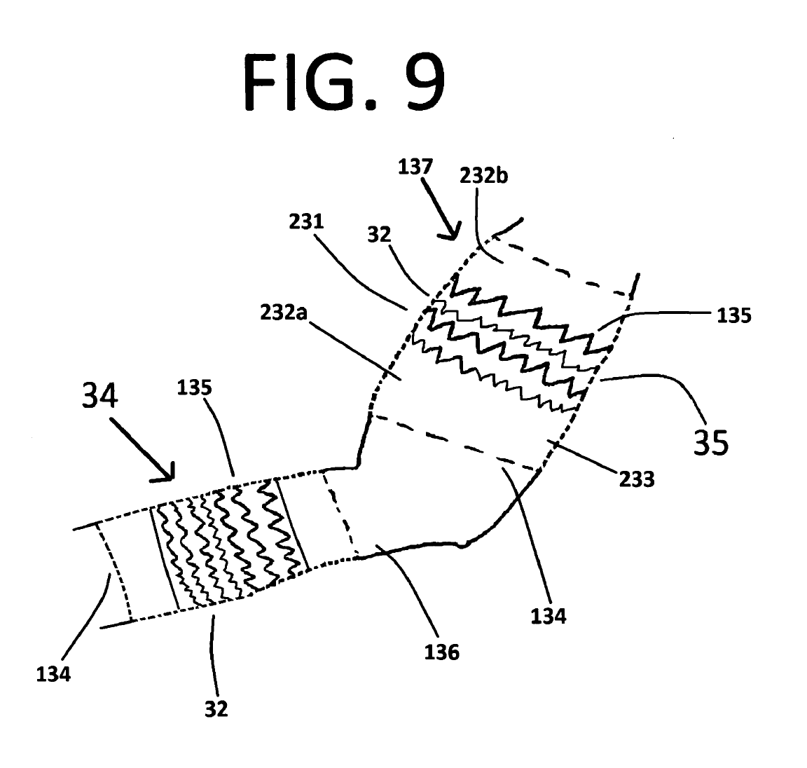

FIG. 9 is a drawing of the embodiment as illustrated in FIG. 3 adhered to the forearm of a user, as well as a similar embodiment adhered to and designed to wrap around the bicep area of a user.

The first embodiment 34 is adhered to the forearm 136 of the user. The second embodiment 137 is attached to the bicep area 35.

Both embodiments provide the same gripping means 135—in this case non-liner depressions 32 with a preferable depth of about seven hundred micrometers, as well as the same adhesive means 134 and adhesive tensile strength. Other adhesives may also comprise low trauma pressure adhesives, if preferred.

The first embodiment 34, as mentioned in FIG. 3, is configured to be of sufficient size and shape to cover the entire forearm inner area, or palmar side surface area of a user's forearm. More specifically, the sports tape overlays at least about eighty percent of the entire forearm inner area, say about four inches for example. A user adhering this embodiment to a forearm will find significantly enhanced gripping capabilities while engaging in sporting activities, such as while running with a football or while making contact with a volleyball.

The embodiment adhered to the user's bicep is made of the same cotton blend, but is four inches wide, so just one wrap or layer is required around the bicep.

The second embodiment 137 is configured to be of sufficient size and shape to cover approximately the entire bicep of a user. The gripping means 135 overlays substantially the entire central portion of the sports tape top surface

As with all embodiments of the present invention, however, the gripping means does not cover the entire sport tape top surface. Centrally located on the sports tape top surface, the gripping means overlays between twenty to fifty percent 231 of the sports tape top surface, leaving at least fifty percent of the top surface smooth and untextured—say about twenty-five percent of each widthside 232a and 232b—and free to stretch by being unconstructed by the more rigid gripping means and high tack coating.

The sports tape also comprises of a moisture resistant coating 233, such as SCOTCH GUARD.

Both embodiments 34, 137 are cut at a length enough to wrap around the arm, say of about seven to nine inches, and thus form the flexible band configurations of tape embodiments, whereby these sports tapes are designed to wrap around a user's hand, forearm or arm area.

Now referring to FIG. 10, shown is the dorsal side of a user's left hand, using the embodiments shown and described in FIG. 5.

The embodiment covering the distal phalange of the pinkie finger comprises an adhesive means 50 on the bottom surface adhered to the pinkie finger and is about one centimeter wide. It has a gripping means 51 in the form of a plurality of depressions 51, such as horizontal circular grooves 51, of about three hundred micrometers in depth throughout the central top surface area of the sports tape, covering approximately fifty percent of the top surface. The remaining approximately fifty percent, about twenty-five percent on one side 153 and about twenty-five percent on the other side 154 adjacent the gripping means, are therefore uncovered by the gripping means thereby allowing the edges of the embodiment to stretch more freely than the portion textured with the more rigid gripping means. The embodiment is comprised of a natural latex material forming the gripping means 51, with a NYLON mesh fibers forming the sports tape 150 to have significant elastic properties, of about one hundred and ten percent and one hundred and eighty percent 150.

The embodiment covering a portion of the ring finger has an adhesive means 52 that is typically used in standard sports tape configurations sports tape configurations along the bottom surface, and has a gripping means 53 comprised of a plurality of depression 53, such as non-linear gripping grooves 53 of about two hundred micrometers in height, throughout forty percent of the central the top surface area of the embodiment, being beveled for example, along the edges for easier application as well as added flexibility. The remaining approximately sixty percent, about thirty percent on one side 155 and about twenty-five percent on the other side 156 adjacent the gripping means, are therefore uncovered by the gripping means thereby allowing the edges of the embodiment to stretch more freely than the portion textured with the more rigid gripping means. The fibers forming this embodiment comprise of a synthetic latex material, to have significant elastic properties, of about one hundred and ten percent and one hundred and twenty percent 250, and is about 1.5 centimeters in width.

The embodiment covering a portion of the middle finger has adhesive means 54 used with skin-contact products, such as, BAND-AID BANDAGES, along the bottom surface of the embodiment, and offers a gripping means 55, such as non-uniform vertical grooves 55 of about % millimeter in depth throughout most of the central top surface portion of the embodiment. The material forming this particular embodiment comprises a rubber material 157 to have significant elastic properties, of about one hundred and ten percent 157, for example, or more, and is about two centimeters in width. The gripping means is provided along sixty percent of the top surface area, leaving the thirty percent closest to its two terminal edges 158, 159, smooth and untextured for easier application and added flexibility. The rubber construction provides glove-like properties and benefits aforementioned, while also enjoying the benefits of tape configurations.

The embodiment covering a portion of the forefinger offers an adhesive means 56 as used in ASSURED LONG LASTING ADHESIVE BANDAGES 56, and provides a gripping means 57 comprised of a plurality of depressions 57, such as vertical grooves 57, for example. This embodiment is made of standard athletic tape construction, such as a cloth encased by a thin, flexible plastic layer 152, such as polyethylene to have significant elastic properties, of about between fifty percent 152 and one hundred percent stretch capability, and is 1.5 centimeters wide. The gripping means is provided along fifty percent of the top surface area, leaving the thirty percent closest to its two terminal edges 156, 251, smooth and untextured for easier application and added flexibility.

There are several widths that the present invention may offer. Embodiments that target the hand may, for example, comprise a preferred width starting at about ½ or one centimeter, but may be as wide as two centimeters—wide enough to essentially cover an entire finger area such as a distal phalange, without having to use multiple layers in an abutting fashion. Additionally, adhesive article embodiments and tape embodiment may have perforations, such as that described in FIG. 4, for example.

Additionally, a portion of the palm area is wrapped with another embodiment 58. Athletes such as football receives may use embodiments in this fashion to better catch or otherwise control a football. This particular embodiment is preferably comprised of a cotton fiber 58, and has a one hundred percent acrylic adhesive means 257 that is latex-free. The embodiment can be configured to last a few days adhered to the skin, beneficial especially if the intended use is at least partly for skin care reasons, such as, for example, if the user would like to maintain tightness in certain muscles. The gripping means 59 comprises of a coating that provides high tack capabilities, such as a PVC coating 59, for example, or other tackifiers that are configured to provide a Shore A Durometer Coefficient of Friction of between 2.0 and 4.5, such as about 3.0, for example. The gripping means 59 is provided along sixty percent of the top surface area 254, leaving the thirty percent closest to its two terminal edges 155, 156, smooth and untextured for easier application and added flexibility. A preferred width of the embodiment is about ½ inch for example 58 and more generally preferably vary in width, from ⅙ inch to 2.5 inches. Furthermore, the embodiment can be porous and breathable, and can additionally be used to support and stabilize injuries to joints, bones and muscles.

This embodiment can alternatively be made of a SPANDEX material, thus providing stretchy properties, of between one hundred and one hundred and eighty percent. Additionally, this and the other embodiments in FIG. 5 can have a paper release sheet (as described in FIG. 2), may be perforated every three inches, and the gripping means may comprise of a plurality of projections (similar to that described in FIG. 2). In still another alternative but similar embodiment, the gripping means may comprise of a plurality of projections such as PVC beads of irregular heights, having a preferred minimum height of about three hundred micrometers, and the material may comprise of cloth. The adhesive and PVC beads could be transferred to the cloth by roller transfer coating or spraying, by stamping or by other standard methods. A preferred thickness of the adhesives may generally be about 0.1 mm after application, applied on the entire bottom surface of the embodiment.

The adhesive means may also comprise of a rubber based adhesive, or non-latex based synthetic adhesive, depending on if the embodiments will be for use in underwater activities.

These embodiments can be water resistant by coating the fibers forming the sports tape with a moisture repellant, for example, a synthetic resin 151.

I claim:

1. A flexible grip-enhancing sports tape adapted to adhere and wrap around a user's finger, hand or arm comprising:
a woven fiber, a non-woven fiber or combinations thereof, forming a flexible and continuous sports tape structure,
said sports tape have a top surface a bottom surface, a first end a second end and wherein the second end is opposite the first end; and
wherein said sports tape is configured to be in a coiled state and is spooled; and
wherein said sports tape has a constant width dimension throughout said sports tape; and
wherein said sports tape further comprises of a gripping means;
wherein said gripping means, disposed on applied to formed on or integral to the top surface of said sports tape creating a topside,
said sports tape further comprising of an adhesive means;
wherein said adhesive means is on said bottom surface, configured to adhere said adhesive article to the human body or to a skin-contact product on the human body.

2. The adhesive article, as claimed in claim 1, wherein the grip-enhancing means comprises:
at least a part of the top surface of said adhesive article,
said grip-enhancing means comprises a high friction surface formed by a high friction material including but not limited to a PVC material, a rubber material, and a neoprene material, and the like, and/or high friction coatings, and/or a plurality of projections, a plurality of depressions, and the like, and/or designs intended to create a high coefficient of friction on the top surface of said adhesive article.

3. The adhesive article, as claimed in claim 1, wherein said fiber or fibers comprises an athletic tape or specialty tape, including but not limited to, kinesiology tape, and is therefore an improvement to athletic tapes and specialty tapes intended to be attached to the human body or to be attached to a skin-contact product.

4. The adhesive article, as claimed in claim 1, wherein said article further comprises:
a therapeutic additive on the bottom surface of said article, a medicated additive, or both, and/or is hypoallergenic,
said therapeutic and medicated additive separated from, or being combined with, the adhesive on said bottom surface,
said adhesive layer comprises pressure sensitive adhesives and other adhesives classified as non-structural.

5. The adhesive article, as claimed in claim 1, further comprising a shock-absorbing member.

6. The adhesive article, as claimed in claim 1, wherein said article further comprises a release sheet which is configured to cover the bottom surface of said article, to keep said adhesive means from drying before use by user.

7. The adhesive article, as claimed in claim 1, wherein said article includes a perforation, said perforation being configured to permit a user to detach a first end from a second end, of said article.

8. A tape, comprising:
a woven fiber (s), a non-woven fiber (s), or combinations thereof,
said tape having a top (outer) surface, a bottom (inner) surface, a first end, a second end, and wherein the second end is opposite the first end,
a grip-enhancing means, disposed on, formed on, integral to, or otherwise in contact with the top surface of said tape creating a topside,
said grip-enhancing means extending to cover at least part of the top surface of said tape,
an adhesive means on the bottom surface of said tape, configured to adhere said tape to the human body or to a skin-contact product on the human body.

9. The tape, as claimed in claim 8, wherein said top surface is printed with data which is readable, including but not limited to a word, a slogan, a phrase, a sentence, or a passage.

10. The tape, as claimed in claim 8, wherein said tape comprises a middle layer comprising a shock-absorbing member consisting of a foam, thereby offering cushiony protective properties to the user.

11. The tape, as claimed in claim 8, wherein said tape comprises:
a therapeutic or medicated additive on the bottom surface of said tape,
said therapeutic and medicated additive separated from, or being combined with, the adhesive on said bottom surface,
said therapeutic additive comprising antiseptics, ointments, and antibiotics, including but not limited to neomycil sulfate, bacitracin, and the like generally known, and/or skin-care agents, including but not limited to, trentinoin, alpha hydroxyl acids, and the like generally known, said adhesive means comprises pressure sensitive adhesives used on athletic tapes intended for application to the skin region, and adhesives used on skin-contact products, including but not limited to BAND-AID, bandages and patches such as SALONPAS.

12. The tape, as claimed in claim 8, wherein said grip-enhancing means comprises a textured surface including but not limited to a plurality of projections, a plurality of depressions, grip-enhancing coatings and/or treatments, and the like, and grip-enhancing patterns, designs, and the like, and/or high friction materials including but not limited to a PVC material, a rubber material, or a neoprene material, and the like, and combinations thereof, creating a high friction surface.

13. The tape, as claimed in claim 8, wherein said tape comprises a release sheet which is configured to cover the bottom surface of said tape to keep said adhesive means from drying before use by user.

14. The tape, as claimed in claim 8, wherein said tape includes a perforation, said perforation being configured to permit a user to detach a first end from a second end, of said tape.

15. The tape, as claimed in claim 8, wherein said tape comprises conventional athletic tapes intended for application to the human body, including but not limited to MCDAVID ATHLETIC TAPE, and specialty tapes intended for application to the human hand or arm, including but not limited to Kinesiology tape, the improvement comprising said grip-enhancing means, and/or said shock-absorbing member.

16. A tape, comprising:
a first material comprising a woven fiber(s), a non-woven fiber(s), or combinations thereof, forming a flexible band,
a second material comprising a woven fiber(s), a non-woven fiber(s), or combinations thereof,
said second material having a top surface at least partially comprising a grip-enhancing means,
said grip-enhancing means comprising a high friction surface, and/or high friction textures, and/or high friction coatings, and/or designs including but not limited to a plurality of projections, a plurality of depressions, and combinations thereof,
said first material comprising an adhesive means on the bottom of said first material,
said adhesive means comprising pressure sensitive adhesives, and other adhesives classified as non-structural and configured for attachment to the human body and/or attachment to a skin-contact product,
said second material being affixed to said first material thus becoming the new top surface of the now reinforced first material,
thereby creating a new athletic tape having a top surface gripping means.

17. The tape, as claimed in claim 16, wherein said new athletic tape comprises a release sheet which is configured to cover the bottom surface of said new athletic tape.

18. The tape, as claimed in claim 16, wherein said first material comprises an athletic tape or specialty tape, including but limited to kinesiology tape, and is therefore an improvement to athletic tapes and specialty tapes that are configured to attach to the human body or to be attached to a skin-contact product including but not limited to, pre-wraps, and the like.

19. The new athletic tape, as claimed in claim 16, wherein said new athletic tape comprises:
standard athletic tape construction including but not limited to, non-woven polyurethane, and the like, and knitted fabrics including but not limited to cotton, and the like, and combinations thereof,
said new athletic tape may include a perforation,
said perforation being configured to permit a user to detach a first end to a second end, of said new athletic tape.

20. The new athletic tape, as claimed in claim 16, wherein said new athletic tape comprises:
materials used in constructing athletic gloves, including but not limited to synthetic leathers, rubbers, plastics, mesh fabrics, spandex, fleeces, latex, and the like, and combinations thereof.

21. The new athletic tape, as claimed in claim 16, further comprising a shock-absorbing member,
said shock-absorbing member comprising of foams including but not limited to open-cell or closed-cell foams, such as BOLLARD Foam, and polyurethane foam, polyvinyl chloride foam, and the like, and/or a plurality of materials with cushiony properties including but not limited to cotton and thick, flexible plastics, thus creating a middle layer within said new athletic tape,
thereby additionally benefiting users by, among other things, enhancing the protection of an injury, and/or protect from injury, by offering a shock-absorbing member substantially within said new athletic tape.

22. The new athletic tape, as claimed in claim 16, wherein said first material further comprises:
a therapeutic or medicated additive on the bottom surface of said first material,
said therapeutic and medicated additive separated from, or being combined with, the adhesive on said bottom surface,
said therapeutic additive comprising antiseptics, ointments, and antibiotics, including but not limited to neomycil sulfate, bacitracin, and the like generally known, and/or skin-care agents, including but not limited to, trentinoin, alpha hydroxyl acids, and the like generally known,
said new athletic tape top surface may additionally be printed with data which is readable, including but not limited to a word, a slogan, a phrase, a sentence, or a passage.
